(19) United States
(12) Patent Application Publication
(10) Pub. No.: US 2023/0414620 A1
(43) Pub. Date: Dec. 28, 2023

(54) TOPICAL RUXOLITINIB FOR TREATING LICHEN PLANUS

(71) Applicants: Incyte Corporation, Wilmington, DE (US); Mayo Foundation for Medical Education and Research, Rochester, MN (US)

(72) Inventors: Aaron R. MANGOLD, Scottsdale, AZ (US); Ahmad B. NAIM, Wilmington, DE (US)

(21) Appl. No.: 18/246,861

(22) PCT Filed: Oct. 1, 2021
(86) PCT No.: PCT/US2021/053149

§ 371 Date: Mar. 28, 2023

Related U.S. Application Data
(60) Provisional application No. 63/086,898, filed on Oct. 2, 2020.

(51) Int. Cl. A61K 31/519; A61K 9/00; A61K 9/107; A61P 17/00

(52) U.S. Cl. CPC A61K 31/519; A61K 9/0014; A61K 9/107; A61P 17/00

(57) ABSTRACT

The present disclosure relates to topical treatment of lichen planus (LP) using ruxolitinib, or a pharmaceutically acceptable salt thereof.

### CROSS-REFERENCE TO RELATED APPLICATIONS

This application claims priority to U.S. Provisional Application No. 63/086,898, filed Oct. 2, 2020, which is hereby incorporated by reference herein in its entirety.

### TECHNICAL FIELD

The present disclosure relates to topical treatment of lichen planus (LP) using ruxolitinib, or a pharmaceutically acceptable salt thereof.

### BACKGROUND

Lichenoid tissue reaction (LTR) includes: lichen planus (LP), cutaneous lupus, dermatomyositis (DM), and graft versus host disease (GVHD). The pathogenesis of LP is incompletely understood; however, it is clearly T-cell mediated without a known auto-antigen. Lichen Planus (LP) is an inflammatory skin condition characterized by purple, polygonal, pruritic, papules and plaques. LP can affect any ectodermally derived tissues including the skin (most commonly), nails, and mucous membranes. LP is thought to affect approximately 1-2% of the general population. Most cutaneous LP resolves within one to two years. Disease duration, in ascending order, is: generalized cutaneous, non-generalized cutaneous, cutaneous and mucosal, mucosal, hypertrophic, and Lichen Planopilaris (hair LP). LP is the prototypical lichen tissue reaction (LTR). Modern theories of LP encompass three major stages: antigen recognition, lymphocyte activation, and keratinocyte apoptosis. A fourth stage, resolution, is a new and emerging topic. The occurrence of triggering factors in a genetically predisposed individual carrying LP-associated genes, results in disease development. During the initiation phase, damage to keratinocytes result in the release of DNA, RNA, and cathelicidin (LL37). These proteins can stimulate plasmacytoid dendritic cells (PDC) via toll like receptors 7, 9 (TLR7,9) which result in the release of interferon alpha (IFNα). IFNα can have both local and distant affects upon myeloid dendritic cells (MDC) as well as keratinocytes. Stimulated MDC interact with CD4+ T-helper cells and with the correct antigen. The antigen remains unknown but may represent a viral derived peptide. Signaling receptors on MDC will stimulate CD4+ T-helper cells via the release of Tumor necrosis factor alpha (TNFα), Interleukin-1 (IL-1), and -12. In addition, cluster of differentiation—(CD40) and CD40 ligand (CD40L) will result in the co-stimulation of the T-helper/MDC interaction. Stimulated CD4+ T-helper cells then release of interferon gamma (IFNγ) and IL-2. These cytokines stimulate CD8+ T-cytotoxic cells. The stimulated CD8+ cells expressing Chemokine receptor-3 (CXCR3) will migrate to the dermal-epidermal junction following the release of chemokines ligands 9(CXCL9), -10,-11. These chemokines are released by stressed and stimulated keratinocytes. The stimulated CD8+ cells will interact with the stressed keratinocytes and can induce apoptosis in with the proper and signaling receptors. This antigen also remains unknown but may be a self-antigen released by local stress. The major kill signals are TNFα, granzyme, and perforin. Fas and Fas ligand (FasL) are expressed on both keratinocytes and lymphocytes. Local CXCR3+DC and T-regulatory cells may also modulate the local lichenoid response.

Based upon critical role of CD8+ T-cytotoxic cells and IFNα and IFNγ signaling in LP, inhibition with topical Janus Kinase (JAK) 1, 2 inhibitors have significant therapeutic potential. The current topical therapeutic options for LP are limited to topical corticosteroids, calcineurin inhibitors, and retinoids. Topical therapies are effective with limited disease but refractory disease is frequently encountered. Specific variants of LP, such as hypertrophic LP and localized cutaneous disease, are often refractory to all topical therapies and require systemic or skin-directed treatment with oral acitretin or methotrexate or light therapy. Therefore, additional topical therapies would be highly desirable in hypertrophic and refractory localized cutaneous LP. As proof of principle, systemic JAK1, 2 inhibition with ruxolitinib was highly effective in steroid refractory graft versus host disease (GVHD) (see e.g., Burchert et al, Leukemia. 2015; 29(10):2062-8. Additionally, there was a recent case report of an exceptional responder to JAK1, 2 inhibition with ruxolitinib in refractory dermatomyositis (DM) (see e.g., Janzen V et al, N Engl J Med. 2014; 371(26):2537-8). As stated above, DM, GVHD, and LP share a similar histological pattern as well as signaling mechanism and, therefore, LP is expected to respond similarly with systemic as well as topical treatment.

Considering these limitations, there is a medical need for new therapeutic options. This application is directed to that need and others.

### SUMMARY

The present application provides, inter alia, methods of treating human patients suffering from lichen planus (LP) with ruxolitinib, or a pharmaceutically acceptable salt thereof.

The present disclosure further provides a ruxolitinib topical formulation for use in any of the methods described herein.

The present disclosure also provides use of a ruxolitinib topical formulation for manufacture of a medicament for use in any of the methods described herein.

The details of one or more embodiments of the invention are set forth in the description below. Other features, objects, and advantages of the invention will be apparent from the description and drawings, and from the claims.

### DETAILED DESCRIPTION

Ruxolitinib is a potent JAK1/JAK2 inhibitor, (R)-3-(4-(7H-pyrrolo[2,3-d]pyrimidin-4-yl)-1H-pyrazol-1-yl)-3-cyclopentylpropanenitrile (INCB018424; ruxolitinib; active ingredient in JAKAFI®), and its pharmaceutically acceptable salts, has previously been described in U.S. Pat. No. 7,598,257, which is incorporated herein by reference in its entirety. Ruxolitinib phosphate was previously described in U.S. Patent Publ. No. 2008/0312259, which is incorporated herein by reference in its entirety.

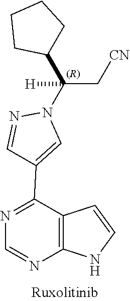

For example, methods are provided for treating cutaneous lichen planus in a human patient in need thereof, comprising administering to an affected skin area of said human patient, a topical formulation comprising ruxolitinib or a pharmaceutically acceptable salt thereof. In other examples, provided are methods are provided for treating cutaneous lichen planus in a human patient in need thereof, comprising administering to an affected skin area of said human patient, a ruxolitinib phosphate cream two times per day. In some embodiments, the ruxolitinib, or the pharmaceutically acceptable salt thereof, is present in an amount of 0.5% to 1.5% (w/w) on a free base basis. In some embodiments, the ruxolitinib, or the pharmaceutically acceptable salt thereof, is present in an amount of 0.75% to 1.5% (w/w) on a free base basis.

The present application provides methods of treating cutaneous lichen planus in a human patient in need thereof, comprising administering to an affected skin area of said human patient, a topical formulation two times per day, wherein said topical formulation comprises 1.5% (w/w) on a free base basis of ruxolitinib phosphate.

The present application provides methods of treating cutaneous lichen planus in a human patient in need thereof, comprising administering to an affected skin area of said human patient, a topical formulation two times per day, wherein said topical formulation comprises 1.5% (w/w) on a free base basis of ruxolitinib phosphate, wherein the patient achieves a reduction in total lesion count from baseline. In some embodiments, the reduction is statistically significant. In some embodiments, the patient achieves a statistically significant reduction in total lesion count from baseline 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient, a cream formulation two times per day, wherein said topical formulation comprises 1.5% (w/w) on a free base basis of ruxolitinib phosphate, wherein the patient achieves an improvement in Index Treatment and Control Lesion by modified Clinical Assessment Scale of Severity for Index Lesion Signs and Symptoms (mCAILS) score from baseline. In some embodiments, the improvement is statistically significant. In some embodiments, the patient achieves a statistically significant reduction in mCAILS score after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient in need thereof, a cream formulation two times per day, wherein said topical formulation comprises 1.5% (w/w) on a free base basis of ruxolitinib phosphate, wherein the patient achieves a reduction in the percentage of the Body Surface Area of the cutaneous lichen planus involvement. In some embodiments, the reduction is statistically significant. In some embodiments, the patient achieves a statistically significant reduction in percentage of the Body Surface Area of the cutaneous lichen planus involvement after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient in need thereof, a cream formulation two times per day, wherein said topical formulation comprises 1.5% (w/w) on a free base basis of ruxolitinib phosphate, wherein the patient achieves a Physician Global Cutaneous Lichen Planus Assessment of (clear), 1 (almost clear), or 2 (significant improvement). In some embodiments, the patient achieves a Physician Global Cutaneous Lichen Planus Assessment of 0 (clear), 1 (almost clear), or 2 (significant improvement) after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient in need thereof, a cream formulation two times per day, wherein said topical formulation comprises 1.5% (w/w) on a free base basis of ruxolitinib phosphate, and wherein the patient achieves a statistically significant improvement in Physician Global Cutaneous Lichen Planus Assessment from baseline. In some embodiments, the patient achieves a statistically significant improvement in Physician Global Cutaneous Lichen Planus Assessment from baseline after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient in need thereof, a cream formulation two times per day, wherein said topical formulation comprises 1.5% (w/w) on a free base basis of ruxolitinib phosphate, and wherein the patient achieves a 1-point improvement from baseline in Physician Global Cutaneous Lichen Planus Assessment. In some embodiments, the patient achieves a 1-point improvement from baseline in Physician Global Cutaneous Lichen Planus Assessment after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient in need thereof, a cream formulation two times per day, wherein said topical formulation comprises 1.5% (w/w) on a free base basis of ruxolitinib phosphate, and wherein the patient achieves a 2-point improvement from baseline in Physician Global Cutaneous Lichen Planus Assessment. In some embodiments, the patient achieves a 2-point improvement from baseline in Physician Global Cutaneous Lichen Planus Assessment 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient in need thereof, a cream formulation two times per day, wherein said topical formulation comprises 1.5% (w/w) on a free base basis of ruxolitinib phosphate, wherein the patient achieves a reduction in pruritus Numerical Rating Scale score from baseline. In some embodiments, the reduction is statistically significant. In some embodiments, the patient achieves a reduction in pruritus Numerical Rating Scale score from baseline after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

The present application provides methods of treating cutaneous lichen planus in a human patient in need thereof, comprising administering to an affected skin area of said human patient in need thereof, a cream formulation two times per day, wherein said topical formulation comprises 1.5% (w/w) on a free base basis of ruxolitinib phosphate, wherein the patient achieves a reduction in Skindex-16 score from baseline. In some embodiments, the reduction is statistically significant. In some embodiments, the patient achieves a reduction in Skindex-16 score from baseline after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering. In some embodiments, the ruxolitinib, or a pharmaceutically acceptable salt thereof, is ruxolitinib phosphate.

In some embodiments, the patient is a man or woman aged≥18 years.

In some embodiments, the patient has clinical and histological features of lichen planus.

In some embodiments, the patient has a Body Surface Area of lichen planus involvement of 2% to 20% at baseline. In some embodiments, the patient has a Body Surface Area of lichen planus involvement of 3% to 20% at baseline. In some embodiments, the patient has a Body Surface Area of lichen planus involvement in a range of 2% to 19%, 2% to 18%, 2% to 17%, 2% to 16%, 2% to 15%, 2% to 14%, 2% to 13%, 2% to 12%, 2% to 11%, 2% to 10%, 2% to 9%, 2% to 8%, 2% to 7%, 2% to 6%, 2% to 5%, 2% to 4%, 2% to 3%, 3% to 20%, 3% to 19%, 3% to 18%, 3% to 17%, 3% to 16%, 3% to 15%, 3% to 14%, 3% to 13%, 3% to 12%, 3% to 11%, 3% to 10%, 3% to 9%, 3% to 8%, 3% to 7%, 3% to 6%, 3% to 5%, 3% to 4%, 4% to 20%, 4% to 19%, 4% to 18%, 4% to 17%, 4% to 16%, 4% to 15%, 4% to 14%, 4% to 13%, 4% to 12%, 4% to 11%, 4% to 10%, 4% to 9%, 4% to 8%, 4% to 7%, 4% to 6%, 4% to 5%, 5% to 20%, 5% to 19%, 5% to 18%, 5% to 17%, 5% to 16%, 5% to 15%, 5% to 14%, 5% to 13%, 5% to 12%, 5% to 11%, 5% to 10%, 5% to 9%, 5% to 8%, 5% to 7%, 5% to 6%, 6% to 20%, 6% to 19%, 6% to 18%, 6% to 17%, 6% to 16%, 6% to 15%, 6% to 14%, 6% to 13%, 6% to 12%, 6% to 11%, 6% to 10%, 6% to 9%, 6% to 8%, 6% to 7%, 7% to 20%, 7% to 19%, 7% to 18%, 7% to 17%, 7% to 16%, 7% to 15%, 7% to 14%, 7% to 13%, 7% to 12%, 7% to 11%, 7% to 10%, 7% to 9%, 7% to 8%, 8% to 20%, 8% to 19%, 8% to 18%, 8% to 17%, 8% to 16%, 8% to 15%, 8% to 14%, 8% to 13%, 8% to 12%, 8% to 11%, 8% to 10%, 8% to 9%, 8% to 8%, 9% to 20%, 9% to 19%, 9% to 18%, 9% to 17%, 9% to 16%, 9% to 15%, 9% to 14%, 9% to 13%, 9% to 12%, 9% to 11%, 9% to 10%, 10% to 20%, 10% to 19%, 10% to 18%, 10% to 17%, 10% to 16%, 10% to 15%, 10% to 14%, 10% to 13%, 10% to 12%, 10% to 11%, 11% to 20%, 11% to 19%, 11% to 18%, 11% to 17%, 11% to 16%, 11% to 15%, 11% to 14%, 11% to 13%, 11% to 12%, 12% to 20%, 12% to 19%, 12% to 18%, 12% to 17%, 12% to 16%, 12% to 15%, 12% to 14%, 12% to 13%, 13% to 20%, 13% to 19%, 13% to 18%, 13% to 17%, 13% to 16%, 13% to 15%, 13% to 14%, 14% to 20%, 14% to 19%, 14% to 18%, 14% to 17%, 14% to 16%, 14% to 15%, 15% to 20%, 15% to 19%, 15% to 18%, 15% to 17%, 15% to 16%, 16% to 20%, 16% to 19%, 16% to 18%, 16% to 17%, 17% to 20%, 17% to 19%, 17% to 18%, 18% to 20%, 18% to 19%, or 19% to 20%.

In some embodiments, the patient has a minimum of 4, preferably 10, lesions of lichen planus. In some embodiments, the patient has a minimum of 4 lesions of lichen planus. In some embodiments, the patient has 10 lesions of lichen planus.

In some embodiments, the patient has a minimum of 10 lesions of lichen planus.

In some embodiments, the patient has a minimum of 20 lesions of lichen planus.

In some embodiments, the patient has a minimum of 30 lesions of lichen planus.

In some embodiments, the patient has a minimum of 40 lesions of lichen planus.

In some embodiments, the patient has a minimum of 50 lesions of lichen planus.

In some embodiments, the patient has a minimum of 60 lesions of lichen planus.

In some embodiments, the patient has a minimum of 70 lesions of lichen planus.

In some embodiments, the patient has had lichen planus for at least 8 months.

In some embodiments, the patient has failed to achieve an adequate response to prior therapy for lichen planus. In some embodiments, the prior therapy is administration of one of the following: one or more topical corticosteroids selected from the group of triamcinolone acetonide, clobetasol propionate, diflorasone diacetate, fluocinolone acetonide, betamethasone valerate, betamethasone dipropionate, amcinonide, desoximetasone, fluocinonide acetonide, halcinonide, hydrocortisone valerate, desonide, hydrocortisone acetate, methylprednisolone acetate, dexamethasone sodium phosphate; topical tacrolimus and pimecrolimus; systemic immunosuppressant; oral metronidazole; oral sulfasalazine; and oral retinoid. In some embodiments, the prior therapy is administration of one of the following: one or more topical corticosteroids selected from the group of triamcinolone acetonide, clobetasol propionate, diflorasone diacetate, fluocinolone acetonide, betamethasone valerate, betamethasone dipropionate, amcinonide, desoximetasone, fluocinonide acetonide, and halcinonide; topical tacrolimus and pimecrolimus; systemic immunosuppressant; oral metronidazole; oral sulfasalazine; oral retinoid. In some embodiments, the prior therapy phototherapy. In some embodiments, the prior therapy is administration of mycophenolate mofetil, methotrexate, azathioprine, or cyclosporine, or a combination thereof.

In some embodiments, the patient achieves a statistically significant reduction in the total lesion count from baseline.

In some embodiments, the patient achieves a statistically significant reduction in the total lesion count from baseline to week 4 of said administering.

In some embodiments, the patient achieves a statistically significant reduction in the total lesion count from baseline to week 8 of said administering.

In some embodiments, the patient achieves a statistically significant reduction in the total lesion count from week 4 to week 8 of said administering.

In some embodiments, the patient a statistically significant reduction in the total lesion count from week 8 to week 12 of said administering.

In some embodiments, the patient achieves a reduction of at least 1 lesions.

In some embodiments, the patient achieves a reduction of at least 2 lesions.

In some embodiments, the patient achieves a reduction of at least 3 lesions.

In some embodiments, the patient achieves a reduction of at least 4 lesions.

In some embodiments, the patient achieves a reduction of at least 5 lesions.

In some embodiments, the patient achieves a reduction of at least 6 lesions.

In some embodiments, the patient achieves a reduction of at least 7 lesions.

In some embodiments, the patient achieves a reduction of at least 8 lesions.

In some embodiments, the patient achieves a reduction of at least 9 lesions. In some embodiments, the patient achieves a reduction of at least 10 lesions.

In some embodiments, the patient achieves a reduction of at least 10 lesions in the total lesion count from baseline to week 4 of said administering.

In some embodiments, the patient achieves a reduction of at least 10 lesions in the total lesion count from baseline to week 8 of said administering.

In some embodiments, the patient achieves a reduction of at least 10 lesions in the total lesion count from week 4 to week 8 of said administering.

In some embodiments, the patient achieves a reduction of at least 10 lesions in the total lesion count from week 8 to week 12 of said administering.

In some embodiments, the patient achieves a reduction of at least 20 lesions.

In some embodiments, the patient achieves a reduction of at least 20 lesions in the total lesion count from baseline to week 4 of said administering.

In some embodiments, the patient achieves a reduction of at least 20 lesions in the total lesion count from baseline to week 8 of said administering.

In some embodiments, the patient achieves a reduction of at least 20 lesions in the total lesion count from week 4 to week 8 of said administering.

In some embodiments, the patient achieves a reduction of at least 20 lesions in the total lesion count from week 8 to week 12 of said administering.

In some embodiments, the patient achieves a reduction of at least 30 lesions.

In some embodiments, the patient achieves a reduction of at least 30 lesions in the total lesion count from baseline to week 4 of said administering.

In some embodiments, the patient achieves a reduction of at least 30 lesions in the total lesion count from baseline to week 8 of said administering.

In some embodiments, the patient achieves a reduction of at least 30 lesions in the total lesion count from week 4 to week 8 of said administering.

In some embodiments, the patient achieves a reduction of at least 30 lesions in the total lesion count from week 8 to week 12 of said administering.

In some embodiments, the patient achieves a reduction of at least 40 lesions.

In some embodiments, the patient achieves a reduction of at least 40 lesions in the total lesion count from baseline to week 4 of said administering.

In some embodiments, the patient achieves a reduction of at least 40 lesions in the total lesion count from baseline to week 8 of said administering.

In some embodiments, the patient achieves a reduction of at least 40 lesions in the total lesion count from week 4 to week 8 of said administering.

In some embodiments, the patient achieves a reduction of at least 40 lesions in the total lesion count from week 8 to week 12 of said administering.

In some embodiments, the patient achieves a reduction of at least 50 lesions.

In some embodiments, the patient achieves a reduction of at least 50 lesions in the total lesion count from baseline to week 4 of said administering.

In some embodiments, the patient achieves a reduction of at least 50 lesions in the total lesion count from baseline to week 8 of said administering.

In some embodiments, the patient achieves a reduction of at least 50 lesions in the total lesion count from week 4 to week 8 of said administering.

In some embodiments, the patient achieves a reduction of at least 50 lesions in the total lesion count from week 8 to week 12 of said administering.

In some embodiments, the patient achieves a statistically significant reduction in modified Clinical Assessment Scale of Severity for Index Lesion Signs and Symptoms score for treatment lesion from baseline.

In some embodiments, the patient achieves a statistically significant reduction in modified Clinical Assessment Scale of Severity for Index Lesion Signs and Symptoms score for treatment lesion from baseline to week 4 of said administering.

In some embodiments, the patient achieves a statistically significant reduction in modified Clinical Assessment Scale of Severity for Index Lesion Signs and Symptoms score for treatment lesion from baseline to week 8 of said administering.

In some embodiments, the patient achieves a statistically significant reduction in modified Clinical Assessment Scale of Severity for Index Lesion Signs and Symptoms score for treatment lesion from week 4 to week 8 of said administering.

In some embodiments, the patient achieves a statistically significant reduction in modified Clinical Assessment Scale of Severity for Index Lesion Signs and Symptoms score for treatment lesion from week 8 to week 12 of said administering.

In some embodiments, the patient achieves at least a 1-point reduction in modified Clinical Assessment Scale of Severity for Index Lesion Signs and Symptoms score for treatment lesion from baseline.

In some embodiments, the patient achieves at least a 1-point reduction in modified Clinical Assessment Scale of Severity for Index Lesion Signs and Symptoms score for treatment lesion from baseline to week 4 of said administering.

In some embodiments, the patient achieves at least a 1-point reduction in modified Clinical Assessment Scale of Severity for Index Lesion Signs and Symptoms score for treatment lesion from baseline to week 8 of said administering.

In some embodiments, the patient achieves at least a 1-point reduction in modified Clinical Assessment Scale of Severity for Index Lesion Signs and Symptoms score for treatment lesion from week 4 to week 8 of said administering.

In some embodiments, the patient achieves at least a 1-point reduction in modified Clinical Assessment Scale of Severity for Index Lesion Signs and Symptoms score for treatment lesion from week 8 to week 12 of said administering.

In some embodiments, the patient achieves at least a 2-point reduction in modified Clinical Assessment Scale of Severity for Index Lesion Signs and Symptoms score for treatment lesion from baseline.

In some embodiments, the patient achieves at least a 2-point reduction in modified Clinical Assessment Scale of Severity for Index Lesion Signs and Symptoms score for treatment lesion from baseline to week 4 of said administering.

In some embodiments, the patient achieves at least a 2-point reduction in modified Clinical Assessment Scale of Severity for Index Lesion Signs and Symptoms score for treatment lesion from baseline to week 8 of said administering.

In some embodiments, the patient achieves at least a 2-point reduction in modified Clinical Assessment Scale of Severity for Index Lesion Signs and Symptoms score for treatment lesion from week 4 to week 8 of said administering.

In some embodiments, the patient achieves at least a 2-point reduction in modified Clinical Assessment Scale of Severity for Index Lesion Signs and Symptoms score for treatment lesion from week 8 to week 12 of said administering.

In some embodiments, the patient achieves at least a 3-point reduction in modified Clinical Assessment Scale of Severity for Index Lesion Signs and Symptoms score for treatment lesion from baseline.

In some embodiments, the patient achieves at least a 3-point reduction in modified Clinical Assessment Scale of Severity for Index Lesion Signs and Symptoms score for treatment lesion from baseline to week 4 of said administering.

In some embodiments, the patient achieves at least a 3-point reduction in modified Clinical Assessment Scale of Severity for Index Lesion Signs and Symptoms score for treatment lesion from baseline to week 8 of said administering.

In some embodiments, the patient achieves at least a 3-point reduction in modified Clinical Assessment Scale of Severity for Index Lesion Signs and Symptoms score for treatment lesion from week 4 to week 8 of said administering.

In some embodiments, the patient achieves at least a 3-point reduction in modified Clinical Assessment Scale of Severity for Index Lesion Signs and Symptoms score for treatment lesion from week 8 to week 12 of said administering.

In some embodiments, the patient achieves at least a 4-point reduction in modified Clinical Assessment Scale of Severity for Index Lesion Signs and Symptoms score for treatment lesion from baseline.

In some embodiments, the patient achieves at least a 4-point reduction in modified Clinical Assessment Scale of Severity for Index Lesion Signs and Symptoms score for treatment lesion from baseline to week 4 of said administering.

In some embodiments, the patient achieves at least a 4-point reduction in modified Clinical Assessment Scale of Severity for Index Lesion Signs and Symptoms score for treatment lesion from baseline to week 8 of said administering.

In some embodiments, the patient achieves at least a 4-point reduction in modified Clinical Assessment Scale of Severity for Index Lesion Signs and Symptoms score for treatment lesion from week 4 to week 8 of said administering.

In some embodiments, the patient achieves at least a 4-point reduction in modified Clinical Assessment Scale of Severity for Index Lesion Signs and Symptoms score for treatment lesion from week 8 to week 12 of said administering.

In some embodiments, the patient achieves at least a 5-point reduction in modified Clinical Assessment Scale of Severity for Index Lesion Signs and Symptoms score for treatment lesion from baseline.

In some embodiments, the patient achieves at least a 5-point reduction in modified Clinical Assessment Scale of Severity for Index Lesion Signs and Symptoms score for treatment lesion from baseline to week 4 of said administering.

In some embodiments, the patient achieves at least a 5-point reduction in modified Clinical Assessment Scale of Severity for Index Lesion Signs and Symptoms score for treatment lesion from baseline to week 8 of said administering.

In some embodiments, the patient achieves at least a 5-point reduction in modified Clinical Assessment Scale of Severity for Index Lesion Signs and Symptoms score for treatment lesion from week 4 to week 8 of said administering.

In some embodiments, the patient achieves at least a 5-point reduction in modified Clinical Assessment Scale of Severity for Index Lesion Signs and Symptoms score for treatment lesion from week 8 to week 12 of said administering.

In some embodiments, the patient achieves at least a 6-point reduction in modified Clinical Assessment Scale of Severity for Index Lesion Signs and Symptoms score for treatment lesion from baseline.

In some embodiments, the patient achieves at least a 6-point reduction in modified Clinical Assessment Scale of Severity for Index Lesion Signs and Symptoms score for treatment lesion from baseline to week 4 of said administering.

In some embodiments, the patient achieves at least a 6-point reduction in modified Clinical Assessment Scale of Severity for Index Lesion Signs and Symptoms score for treatment lesion from baseline to week 8 of said administering.

In some embodiments, the patient achieves at least a 6-point reduction in modified Clinical Assessment Scale of Severity for Index Lesion Signs and Symptoms score for treatment lesion from week 4 to week 8 of said administering.

In some embodiments, the patient achieves at least a 6-point reduction in modified Clinical Assessment Scale of Severity for Index Lesion Signs and Symptoms score for treatment lesion from week 8 to week 12 of said administering.

In some embodiments, the patient achieves at least a 7-point reduction in modified Clinical Assessment Scale of Severity for Index Lesion Signs and Symptoms score for treatment lesion from baseline.

In some embodiments, the patient achieves at least a 7-point reduction in modified Clinical Assessment Scale of Severity for Index Lesion Signs and Symptoms score for treatment lesion from baseline to week 4 of said administering.

In some embodiments, the patient achieves at least a 7-point reduction in modified Clinical Assessment Scale of Severity for Index Lesion Signs and Symptoms score for treatment lesion from baseline to week 8 of said administering.

In some embodiments, the patient achieves at least a 7-point reduction in modified Clinical Assessment Scale of Severity for Index Lesion Signs and Symptoms score for treatment lesion from week 4 to week 8 of said administering.

In some embodiments, the patient achieves at least a 7-point reduction in modified Clinical Assessment Scale of Severity for Index Lesion Signs and Symptoms score for treatment lesion from week 8 to week 12 of said administering.

In some embodiments, the patient achieves a statistically significant difference between the modified Clinical Assessment Scale of Severity for Index Lesion Signs and Symptoms score reductions for treatment lesion and control lesion.

In some embodiments, the patient achieves at least 1-point difference between the modified Clinical Assessment Scale of Severity for Index Lesion Signs and Symptoms score reductions for treatment lesion and control lesion.

In some embodiments, the patient achieves at least 2-point difference between the modified Clinical Assessment Scale of Severity for Index Lesion Signs and Symptoms score reductions for treatment lesion and control lesion.

In some embodiments, the patient achieves at least 3-point difference between the modified Clinical Assessment Scale of Severity for Index Lesion Signs and Symptoms score reductions for treatment lesion and control lesion.

In some embodiments, the patient achieves at least 4-point difference between the modified Clinical Assessment Scale of Severity for Index Lesion Signs and Symptoms score reductions for treatment lesion and control lesion.

In some embodiments, the patient achieves at least 5-point difference between the modified Clinical Assessment Scale of Severity for Index Lesion Signs and Symptoms score reductions for treatment lesion and control lesion.

In some embodiments, the patient achieves at least 6-point difference between the modified Clinical Assessment Scale of Severity for Index Lesion Signs and Symptoms score reductions for treatment lesion and control lesion.

In some embodiments, the patient achieves at least 7-point difference between the modified Clinical Assessment Scale of Severity for Index Lesion Signs and Symptoms score reductions for treatment lesion and control lesion.

In some embodiments, the patient achieves a reduction in the percentage of the Body Surface Area of the cutaneous lichen planus involvement. In some embodiments, the reduction is statistically significant.

In some embodiments, the patient achieves a statistically significant reduction in the percentage of the Body Surface Area score of the cutaneous lichen planus involvement from baseline to week 4 of said administering.

In some embodiments, the patient achieves a statistically significant reduction in the percentage of the Body Surface Area score of the cutaneous lichen planus involvement from baseline to week 8 of said administering.

In some embodiments, the patient achieves a statistically significant reduction in the percentage of the Body Surface Area score of the cutaneous lichen planus involvement from week 4 to week 8 of said administering.

In some embodiments, the patient achieves a statistically significant reduction in the percentage of the Body Surface Area score of the cutaneous lichen planus involvement from week 8 to week 12 of said administering.

In some embodiments, the patient achieves at least a 10% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 20% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 30% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 40% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 50% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 60% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 70% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 80% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves at least a 90% reduction in the percentage of the Body Surface Area of the cutaneous lichen planus involvement. In some embodiments, the reduction in the percentage of the Body Surface Area of the cutaneous lichen planus involvement is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves Physician Global Cutaneous Lichen Planus Assessment of 0 (clear), 1 (almost clear), or 2 (significant improvement). In some embodiments, the Physician Global Cutaneous Lichen Planus Assessment of 0 (clear), 1 (almost clear), or 2 (significant improvement) is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves Physician Global Cutaneous Lichen Planus Assessment of 0 (clear). In some embodiments, the Physician Global Cutaneous Lichen Planus Assessment of 0 (clear) is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves Physician Global Cutaneous Lichen Planus Assessment of 1 (almost clear). In some embodiments, the Physician Global Cutaneous Lichen Planus Assessment of 1 (almost clear) is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves Physician Global Cutaneous Lichen Planus Assessment of 2 (significant improvement). In some embodiments, the Physician Global Cutaneous Lichen Planus Assessment of 2 (significant improvement) is achieved after 1 day, or 2 days, or 3 days, or 4 days, or 5 days, or 6 days, or 7 days, or 8 days, or 9 days, or 10 days, or 11 days, or 12 days, or 13 days, or 14 days, or 3 weeks, or 4 weeks, or 5 weeks, or 6 weeks, or 7 weeks, or 8 weeks of said administering.

In some embodiments, the patient achieves a reduction in pruritus Numerical Rating Scale score from baseline. In some embodiments, the reduction is statistically significant.

In some embodiments, the patient achieves a statistically significant reduction in in pruritus Numerical Rating Scale score from baseline to week 4 of said administering.

In some embodiments, the patient achieves a statistically significant reduction in in pruritus Numerical Rating Scale score from baseline to week 8 of said administering.

In some embodiments, the patient achieves a statistically significant reduction in in pruritus Numerical Rating Scale score from week 4 to week 8 of said administering.

In some embodiments, the patient achieves a statistically significant reduction in in pruritus Numerical Rating Scale score from week 8 to week 12 of said administering.

In some embodiments, the patient achieves at least 1-point reduction in pruritus Numerical Rating Scale score from baseline.

In some embodiments, the patient achieves at least 1-point reduction in in pruritus Numerical Rating Scale score from baseline to week 4 of said administering.

In some embodiments, the patient achieves at least 1-point reduction in in pruritus Numerical Rating Scale score from baseline to week 8 of said administering.

In some embodiments, the patient achieves at least 1-point reduction in in pruritus Numerical Rating Scale score from week 4 to week 8 of said administering.

In some embodiments, the patient achieves at least 1-point reduction in in pruritus Numerical Rating Scale score from week 8 to week 12 of said administering.

In some embodiments, the patient achieves at least 2-point reduction in pruritus Numerical Rating Scale score from baseline.

In some embodiments, the patient achieves at least 2-point reduction in in pruritus Numerical Rating Scale score from baseline to week 4 of said administering.

In some embodiments, the patient achieves at least 2-point reduction in in pruritus Numerical Rating Scale score from baseline to week 8 of said administering.

In some embodiments, the patient achieves at least 2-point reduction in in pruritus Numerical Rating Scale score from week 4 to week 8 of said administering.

In some embodiments, the patient achieves at least 2-point reduction in in pruritus Numerical Rating Scale score from week 8 to week 12 of said administering.

In some embodiments, the patient achieves at least 3-point reduction in pruritus Numerical Rating Scale score from baseline. In some embodiments, the patient achieves at least 3-point reduction in in pruritus Numerical Rating Scale score from baseline to week 4 of said administering.

In some embodiments, the patient achieves at least 3-point reduction in in pruritus Numerical Rating Scale score from baseline to week 8 of said administering.

In some embodiments, the patient achieves at least 3-point reduction in in pruritus Numerical Rating Scale score from week 4 to week 8 of said administering.

In some embodiments, the patient achieves at least 3-point reduction in in pruritus Numerical Rating Scale score from week 8 to week 12 of said administering.

In some embodiments, the patient achieves at least 4-point reduction in pruritus Numerical Rating Scale score from baseline.

In some embodiments, the patient achieves at least 4-point reduction in in pruritus Numerical Rating Scale score from baseline to week 4 of said administering.

In some embodiments, the patient achieves at least 4-point reduction in in pruritus Numerical Rating Scale score from baseline to week 8 of said administering.

In some embodiments, the patient achieves at least 4-point reduction in in pruritus Numerical Rating Scale score from week 4 to week 8 of said administering.

In some embodiments, the patient achieves at least 4-point reduction in in pruritus Numerical Rating Scale score from week 8 to week 12 of said administering.

In some embodiments, the patient achieves a reduction in the Skindex-16 score. In some embodiments, the reduction is statistically significant.

In some embodiments, the patient achieves a statistically significant reduction in Skindex-16 score from baseline to week 4 of said administering.

In some embodiments, the patient achieves a statistically significant reduction in Skindex-16 score from baseline to week 8 of said administering.

In some embodiments, the patient achieves a statistically significant reduction in Skindex-16 score from week 4 to week 8 of said administering.

In some embodiments, the patient achieves a statistically significant reduction in Skindex-16 score from week 8 to week 12 of said administering.

In some embodiments, the patient achieves at least 10-point reduction in the Skindex-16 score.

In some embodiments, the patient achieves at least 10-point reduction in Skindex-16 score from baseline to week 4 of said administering.

In some embodiments, the patient achieves at least 10-point reduction in Skindex-16 score from baseline to week 8 of said administering.

In some embodiments, the patient achieves at least 10-point reduction in Skindex-16 score from week 4 to week 8 of said administering.

In some embodiments, the patient achieves at least 10-point reduction in Skindex-16 score from week 8 to week 12 of said administering.

In some embodiments, the patient achieves at least 20-point reduction in the Skindex-16 score.

In some embodiments, the patient achieves at least 20-point reduction in Skindex-16 score from baseline to week 4 of said administering.

In some embodiments, the patient achieves at least 20-point reduction in Skindex-16 score from baseline to week 8 of said administering.

In some embodiments, the patient achieves at least 20-point reduction in Skindex-16 score from week 4 to week 8 of said administering.

In some embodiments, the patient achieves at least 20-point reduction in Skindex-16 score from week 8 to week 12 of said administering.

In some embodiments, the patient achieves at least 30-point reduction in the Skindex-16 score.

In some embodiments, the patient achieves at least 30-point reduction in Skindex-16 score from baseline to week 4 of said administering.

In some embodiments, the patient achieves at least 30-point reduction in Skindex-16 score from baseline to week 8 of said administering.

In some embodiments, the patient achieves at least 30-point reduction in Skindex-16 score from week 4 to week 8 of said administering.

In some embodiments, the patient achieves at least 30-point reduction in Skindex-16 score from week 8 to week 12 of said administering.

In some embodiments, the patient achieves at least 40-point reduction in the Skindex-16 score.

In some embodiments, the patient achieves at least 40-point reduction in Skindex-16 score from baseline to week 4 of said administering.

In some embodiments, the patient achieves at least 40-point reduction in Skindex-16 score from baseline to week 8 of said administering.

In some embodiments, the patient achieves at least 40-point reduction in Skindex-16 score from week 4 to week 8 of said administering.

In some embodiments, the patient achieves at least 40-point reduction in Skindex-16 score from week 8 to week 12 of said administering.

In some embodiments, the administering is maintained for at least 4 week.

In some embodiments, the administering is maintained for at least 8 weeks.

In some embodiments, the administering is maintained for at least 12 weeks.

In some embodiments, the two administrations per day are at least 8 hours apart.

In some embodiments, the two administrations per day are about 12 hours apart.

In some embodiments, the topical formulation is a cream comprising the ruxolitinib, or pharmaceutically acceptable salt thereof.

In some embodiments, the cream formulation is an oil-in-water emulsion comprising the ruxolitinib, or pharmaceutically acceptable salt thereof.

In some embodiments, the cream formulation is an oil-in-water emulsion comprising said 1.5% (w/w) on a free base basis of ruxolitinib phosphate.

In some embodiments, the cream formulation has a pH from about 2.8 to about 3.6.

In some embodiments, the cream formulation has a pH from about 2.8 to about 3.9.

In some embodiments, the cream formulation is a solubilized cream.

In some embodiments, further comprising administering to the patient an additional therapeutic agent.

The present application provides, inter alia, methods of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof;
wherein the patient:
   is a man or woman aged≥18 years;
   has clinical and histological features of lichen planus; and
   has a Body Surface Area of lichen planus involvement of 2% to 20% at baseline.

The present application provides, inter alia, methods of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof;
wherein the patient:
   is a man or woman aged≥18 years;
   has clinical and histological features of lichen planus;
   has a Body Surface Area of lichen planus involvement of up to 20% at baseline; and
   has a minimum of 4 lesions of lichen planus; and
wherein the patient achieves a reduction of at least 50 lesions in total lesion count from baseline.

The present application provides, inter alia, methods of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof;
wherein the patient:
   is a man or woman aged≥18 years;
   has clinical and histological features of lichen planus;
   has a Body Surface Area of lichen planus involvement of up to 20% at baseline; and
   has 10 lesions of lichen planus; and
wherein the patient achieves a reduction of at least 50 lesions in total lesion count from baseline.

The present application provides, inter alia, methods of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof;
wherein the patient:
   is a man or woman aged≥18 years;
   has clinical and histological features of lichen planus;
   has a Body Surface Area of lichen planus involvement of 2% to 20% at baseline; and
   has a minimum of 4 lesions of lichen planus; and
wherein the patient achieves a reduction of at least 50 lesions in total lesion count from baseline.

The present application provides, inter alia, methods of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof;
wherein the patient:
   is a man or woman aged≥18 years;
   has clinical and histological features of lichen planus;
   has a Body Surface Area of lichen planus involvement of 2% to 20% at baseline; and
   has 10 lesions of lichen planus; and
wherein the patient achieves a reduction of at least 50 lesions in total lesion count from baseline.

The present application provides, inter alia, methods of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof;
wherein the patient:
   is a man or woman aged≥18 years;
   has clinical and histological features of lichen planus;
   has a Body Surface Area of lichen planus involvement of up to 20% at baseline; and
   has a minimum of 10 lesions of lichen planus; and
wherein the patient achieves a reduction of at least 50 lesions in total lesion count from baseline.

The present application provides, inter alia, methods of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof;
wherein the patient:
   is a man or woman aged≥18 years;
   has clinical and histological features of lichen planus;
   has a Body Surface Area of lichen planus involvement of 2% to 20% at baseline; and
   has a minimum of 10 lesions of lichen planus; and
wherein the patient achieves a reduction of at least 50 lesions in total lesion count from baseline.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

### Definitions

As used herein, “an affected skin area” refers to an area of the patient's skin having lichen planus lesions.

As used herein, “ruxolitinib phosphate” means the phosphoric acid salt of ruxolitinib, wherein the ruxolitinib and phosphoric acid are in a 1:1 ratio.

In some embodiments, “cream” means an emulsion, semisolid dosage form for application to the skin.

When the methods refer to “baseline to week 4” or “baseline to week 8” of the administering, this refers to the time period following the first dose of the cream formulation wherein there is no interruption in the administration. For example, if the method refers to a reduction in pruritus NRS score from baseline to week 4 for a patient administered the cream formulation BID, this means the pruritus NRS score was assessed after 8 weeks of BID administration of the cream formulation following the first dose of the cream formulation with no days being skipped.

When the methods refer to “from week 4 to week 8” of the administering, this refers to the time period from the end of week 4 until the end of week 8 of administering the cream formulation, wherein there is no interruption in the administration. For example, if the method refers to a reduction in pruritus NRS score from week 4 to week 8 for a patient administered the cream formulation BID, this means the pruritus NRS score was assessed respectively at the end of week 4 and week 8 of BID administration of the cream formulation.

When the methods refer to “from week 8 to week 12” of the administering, this refers to the time period from the end of week 8 until the end of week 12 of administering the cream formulation, wherein there is no interruption in the administration. For example, if the method refers to a reduction in pruritus NRS score from week 8 to week 12 for a patient administered the cream formulation BID, this means the pruritus NRS score was assessed respectively at the end of week 8 and week 12 of BID administration of the cream formulation.

As used herein, “% BSA” refers to percentage of total Body Surface Area affected by lichen planus (LP). It can be determined to the nearest 0.1% (“handprint”) using, as guides, the palm with fingers as 1% and the thumb as 0.1%. In some embodiments, the % BSA excludes the scalp. In some embodiments, the % BSA excludes the face.

As used herein, “CAILS” refers to clinical assessment scale of severity for index lesion signs and symptoms, which has a 9-point scale as shown in the table below.

| Score | Grade |
|---|---|
| 0 | No evidence of sign or symptom |
| 1 | Intermediate interval |
| 2 | Mild: less than average presentation of sign or symptom |
| 3 | Intermediate interval |
| 4 | Moderate: average disease presentation of sign or symptom |
| 5 | Intermediate interval |
| 6 | Severe: greater than 25% worse than average severity of |
| | sign or symptom |
| 7 | Intermediate interval |
| 8 | Very severe: the near worst severity sign or symptom |

As used herein, the term “modified CAILS” or “mCAILS” score refers to summation of Erythema (0-8), Scaling (0-8), Plaque elevation (0-8), and Size (0-18). The modified CAILS score is 0-42 and the higher score indicated higher severity. A scale of 0 to 18 was used to grade lesion size by square centimeter (0, 0 [no measurable area]; 1, >0 and ≤4; 2, >4 and ≤10; 3, >10 and ≤16; 4, >16 and ≤25; 5, >25 and ≤35; 6, >35 and ≤45; 7, >45 and ≤55; 8, >55 and ≤70; 9, >70 and ≤90; 10, >90 and ≤110; 11, >110 and ≤130; 12, >130 and ≤155; 13, >155 and ≤180; 14, >180 and ≤210; 15, >210 and ≤240; 16, >240 and ≤270; 17, >270 and ≤300; and 18, >300). The area of the lesion will be measured with digital planimetry (see e.g., Olsen et al, Journal of clinical oncology: official journal of the American Society of Clinical Oncology. 011; 29(18):2598-607; Duvic et al, Arch Dermatol. 2001; 137(5):581-93); Rogers et al, J Diabetes Sci Technol. 2010; 4(4):799-802).

As used herein, “PGA” refers to Physician Global Assessment. For example, the PGA has been used herein to evaluate the severity of cutaneous lichen planus and it is referred as the Physician Global Cutaneous Lichen Planus Assessment (PhCLPGA), which has a 7-point scale as shown in the table below.

| Score | Severity | Description |
|---|---|---|
| 0 | Clear | No evidence of disease (100% improvement) CCR |
| 1 | Almost clear | Very significant clearance (≥90% to <100%) PR |
| 2 | Marked Improvement | Significant improvement (≥75% to <90%)PR |
| 3 | Moderate improvement | Intermediate between slight and marked (≥50% |
| | | to <75%) PR |
| 4 | Slight improvement | Some improvement (≥25% to <50%); however, |
| | | significant evidence of disease remains SD |
| 5 | No change | Disease has not changed from baseline condition |
| | | (+/−<25%) SD |
| 6 | Worse | disease is worse than at baseline evaluation by |
| | | (≥25%) or more PD |

CCR—Complete clinical response
PR—Partial response
SD—Stable disease
PD—Progressive Disease

As used herein, “pruritus NRS score” or “itch NRS score” refers to pruritus Numerical Rating Scale. The pruritus NRS is a daily patient-reported measure (24-hour recall) of itch intensity. Subjects will be asked to rate the itching severity because of their LP by selecting a number from 0 (no itch) to 10 (worst imaginable itch) as shown in the table below that best describes their worst level of itching in the past 24 hours. In a non-limiting example, patients can be issued a hand-held device (eDiary) on which to record itch severity. The patient can be instructed to complete the eDiary each night.

| Pruritus NRS score | Itchiness |
|---|---|
| 0 | No itch |
| 1-4 | 1-4 Mild itch |
| 4-7 | 4-7 Moderate itch |
| 7-9 | 7-9 Severe itch |
| 10 | 10- Very severe itch |

As used herein, “Skindex 16” score refers to the total Skindex-16 score. Skindex 16 is a 16-item patient-completed assessment to rate skin conditions using numerical analogue scales (0=never bothered to 6=always bothered). Responses to the Skindex-16 are categorized into three subscales: symptom, emotional and functional as shown in the table below. The total Skindex-16 score is 0-96.

| Subscale | | |
|---|---|---|
| Categories | Subscale (0 = never bothered to 6 = always bothered) | |
| Symptom | 1. | Skin itching |
| Subscale | 2. | Skin burning or stinging |
| | 3. | Skin hurting |
| | 4. | Skin irritated |
| Emotional | 5. | Persistence or recurrence of condition |
| Subscale | 6. | Worry about condition |
| | 7. | Appearance of skin |
| | 8. | Frustration about skin |
| | 9. | Embarrassment about skin |
| | 10. | Annoyed about skin |
| | 11. | Feeling depressed |
| Functional | 12. | Effect of skin on interaction with others |
| Subscale | 13. | Effect of skin on desire to be with people |
| | 14. | Skin making it hard to show affection |
| | 15. | Effect of skin on daily activity |
| | 16. | Skin making it hard to work/have enjoyment |

As used herein, “BID” refers to two times per day.

As used herein, “statistically significant” means a p-value of <0.05 (preferably < and most preferably <0.0001).

As used herein, the phrase “pharmaceutically acceptable” means those compounds, materials, compositions, and/or dosage forms, which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. In some embodiments, the pharmaceutically acceptable salt is a phosphoric acid salt, a sulfuric acid salt, or a maleic acid salt.

As used herein, the term “emulsifier component” refers, in one aspect, to a substance, or mixtures of substances that maintains an element or particle in suspension within a fluid medium. In some embodiments, the emulsifier component allows an oil phase to form an emulsion when combined with water. In some embodiments, the emulsifier component refers to one or more non-ionic surfactants.

As used herein, the term “occlusive agent component” refers to a hydrophobic agent or mixtures of hydrophobic agents that form an occlusive film on skin that reduces transepidermal water loss (TEWL) by preventing evaporation of water from the stratum corneum.

As used herein, the term “stiffening agent component” refers to a substance or mixture of substances that increases the viscosity and/or consistency of the cream or improves the rheology of the cream.

As used herein, the term “emollient component” refers to an agent that softens or soothes the skin or soothes an irritated internal surface.

As used herein, the term “stabilizing agent component” refers to a substance or mixture of substances that improves the stability of the cream and/or the compatibility of the components in the cram. In some embodiments, the stabilizing agent component prevents agglomeration of the emulsion and stabilizes the droplets in the oil-in-water emulsion.

As used herein, the term “solvent component” is a liquid substance or mixture of liquid substances capable of dissolving ruxolitinib (or its salt) or other substances in the cream. In some embodiments, the solvent component is a liquid substance or mixture of liquid substances in which ruxolitinib, or its pharmaceutically acceptable salt, has reasonable solubility. For example, solubilities of ruxolitinib (free base) or its phosphate salt (1:1 salt) are reported in Table 1. In some embodiments, a solvent is a substance or mixture thereof, in which ruxolitinib, or its pharmaceutically acceptable salt (whichever is used), has a solubility of at least about 10 mg/mL or greater, at least about 15 mg/mL or greater, or at least about 20 mg/mL or greater, when measured as described in Example 2.

As used herein, the phrase “antimicrobial preservative component” is a substance or mixtures of substances, which inhibits microbial growth in the cream.

As used herein, the phrase “chelating agent component” refers to a compound or mixtures of compounds that has the ability to bind strongly with metal ions.

As used herein, “% by weight of the emulsion” means the percent concentration of the component in the emulsion is on weight/weight basis. For example, 1% w/w of component A=[(mass of component A)/(total mass of the emulsion)]×100.

As used herein, “% by weight of the emulsion on a free base basis” of ruxolitinib, or pharmaceutically acceptable salt thereof” means that the % w/w is calculated based on the weight of ruxolitinib in the total emulsion. For example, “1.5% w/w on a free base basis” of ruxolitinib phosphate means that for 100 grams of total formulation, there are 1.98 grams of ruxolitinib phosphate in the emulsion (which equates to 1.5 grams of the free base, ruxolitinib).

As used herein, “% by weight of the formulation on a free base basis” of ruxolitinib, or pharmaceutically acceptable salt thereof” means that the % w/w is calculated based on the weight of ruxolitinib in the total formulation. For example, “1.5% w/w on a free base basis” of ruxolitinib phosphate means that for 100 grams of total formulation, there are 0.66 grams of ruxolitinib phosphate in the formulation (which equates to 1.5 grams of the free base, ruxolitinib).

As used herein, the term “component” can mean one substance or a mixture of substances.

As used herein, the term “fatty acid” refers to an aliphatic acid that is saturated or unsaturated. In some embodiments, the fatty acid is in a mixture of different fatty acids. In some embodiments, the fatty acid has between about eight to about thirty carbons on average. In some embodiments, the fatty acid has about 12 to 20, 14-20, or 16-18 carbons on average. Suitable fatty acids include, but are not limited to, cetyl acid, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, behenic acid, isobehenic acid, and arachidonic acid, or mixtures thereof.

As used herein, the term “fatty alcohol” refers to an aliphatic alcohol that is saturated or unsaturated. In some embodiments, the fatty alcohol is in a mixture of different fatty alcohols. In some embodiments, the fatty alcohol has between about 12 to about 20, about 14 to about 20, or about 16 to about 18 carbons on average. Suitable fatty alcohols include, but are not limited to, stearyl alcohol, lauryl alcohol, palmityl alcohol, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “polyalkylene glycol”, employed alone or in combination with other terms, refers to a polymer containing oxyalkylene monomer units, or copolymer of different oxyalkylene monomer units, wherein the alkylene group has 2 to 6, 2 to 4, or 2 to 3 carbon atoms. As used herein, the term “oxyalkylene”, employed alone or in combination with other terms, refers to a group of formula —O-alkylene-. In some embodiments, the polyalkylene glycol is polyethylene glycol.

As used herein, the term, “sorbitan fatty ester” includes products derived from sorbitan or sorbitol and fatty acids and, optionally, poly(ethylene glycol) units, including sorbitan esters and polyethoxylated sorbitan esters. In some embodiments, the sorbitan fatty ester is a polyethoxylated sorbitan ester.

As used herein, the term “sorbitan ester” refers to a compound, or mixture of compounds, derived from the esterification of sorbitol and at least one fatty acid. Fatty acids useful for deriving the sorbitan esters include, but are not limited to, those described herein. Suitable sorbitan esters include, but are not limited to, the Span™ series (available from Uniqema), which includes Span 20 (sorbitan monolaurate), 40 (sorbitan monopalmitate), 60 (sorbitan monostearate), 65 (sorbitan tristearate), 80 (sorbitan monooleate), and 85 (sorbitan trioleate). Other suitable sorbitan esters include those listed in R. C. Rowe and P. J. Shesky, Handbook of pharmaceutical excipients, (2006), ed., which is incorporated herein by reference in its entirety.

As used herein, the term “polyethoxylated sorbitan ester” refers to a compound, or mixture thereof, derived from the ethoxylation of a sorbitan ester. The polyoxethylene portion of the compound can be between the fatty ester and the sorbitan moiety. As used herein, the term “sorbitan ester” refers to a compound, or mixture of compounds, derived from the esterification of sorbitol and at least one fatty acid. Fatty acids useful for deriving the polyethoyxlated sorbitan esters include, but are not limited to, those described herein. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 4 to about 80 oxyethylene units. In some embodiments, the polyoxyethylene portion of the compound or mixture has about 4 to about 40 oxyethylene units. In some embodiments, the polyoxyethylene portion of the compound or mixture has about 4 to about 20 oxyethylene units. Suitable polyethoxylated sorbitan esters include, but are not limited to the Tween™ series (available from Uniqema), which includes Tween 20 (POE(20) sorbitan monolaurate), 21 (POE(4) sorbitan monolaurate), (POE(20) sorbitan monopalmitate), 60 (POE(20) sorbitan monostearate), 60K (POE(20) sorbitan monostearate), 61 (POE(4) sorbitan monostearate), 65 (POE(20) sorbitan tristearate), 80 (POE(20) sorbitan monooleate), 80K (POE(20) sorbitan monooleate), 81 (POE(5) sorbitan monooleate), and 85 (POE(20) sorbitan trioleate). As used herein, the abbreviation “POE” refers to polyoxyethylene. The number following the POE abbreviation refers to the number of oxyethylene repeat units in the compound. Other suitable polyethoxylated sorbitan esters include the polyoxyethylene sorbitan fatty acid esters listed in R. C. Rowe and P. J. Shesky, Handbook of pharmaceutical excipients, (2006), 5th ed., which is incorporated herein by reference in its entirety. In some embodiments, the polyethoxylated sorbitan ester is a polysorbate. In some embodiments, the polyethoxylated sorbitan ester is polysorbate 20.

As used herein, the term “glyceryl fatty esters” refers to mono-, di- or triglycerides of fatty acids. The glyceryl fatty esters may be optionally substituted with sulfonic acid groups, or pharmaceutically acceptable salts thereof. Suitable fatty acids for deriving glycerides of fatty acids include, but are not limited to, those described herein. In some embodiments, the glyceryl fatty ester is a mono-glyceride of a fatty acid having 12 to 18 carbon atoms. In some embodiments, the glyceryl fatty ester is glyceryl stearate.

As used herein, the term “triglycerides” refers to a triglyceride of a fatty acid. In some embodiments, the triglyceride is medium chain triglycerides.

As used herein, the term “alkylene glycol” refers to a group of formula —O-alkylene-, wherein the alkylene group has 2 to 6, 2 to 4, or 2 to 3 carbon atoms. In some embodiments, the alkylene glycol is propylene glycol (1,2-propanediol).

As used herein, the term “polyethylene glycol” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present disclosure can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer.

As used herein, the term “contacting” refers to the bringing together of indicated moieties in an in vitro system or an in vivo system. For example, “contacting” a JAK with a compound of the invention includes the administration of a compound of the present application to an individual or patient, such as a human, having a JAK, as well as, for example, introducing a compound of the invention into a sample containing a cellular or purified preparation containing the JAK.

As used herein, “contains” is equivalent to “comprises”.

As used herein, the term “subject,” “individual,” or “patient,” used interchangeably, refers to humans. In some embodiments, the “subject,” “individual,” or “patient” is in need of said treatment.

In some embodiments, the compounds, or pharmaceutically acceptable salts thereof, or pharmaceutical formulations thereof, topical formulations thereof, as described herein are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease; or (3) preventing the disease, condition or disorder in an individual who may be predisposed to the disease, condition or disorder but does not yet experience or display the pathology or symptomatology of the disease. In some embodiments, treating refers to inhibiting or ameliorating the disease. In some embodiments, treating is preventing the disease.

In some embodiments, the components are present in exactly the ranges specified (e.g., the term “about” is not present). In some embodiments, “about” means plus or minus 10% of the value.

### Combination Therapies

The methods described herein can further comprise administering one or more additional therapeutic agents. The one or more additional therapeutic agents can be administered to a patient simultaneously or sequentially.

In some embodiments, the additional therapeutic agent is an antibiotic. In some embodiments, the antibiotic is clindamycin, doxycycline, minocycline, trimethoprim-sulfamethoxazole, erythromycin, metronidazole, rifampin, moxifloxacin, dapsone, or a combination thereof. In some embodiments, the antibiotic is clindamycin, doxycycline, minocycline, trimethoprim-sulfamethoxazole, or erythromycin in combination with metronidazole. In some embodiments, the antibiotic is a combination of rifampin, moxifloxacin, and metronidazole. In some embodiments, the antibiotic is a combination of moxifloxacin and rifampin.

In some embodiments, the additional therapeutic agent is a retinoid. In some embodiments, the retinoid is adapalene, etretinate, acitretin, or isotretinoin.

In some embodiments, the additional therapeutic agent is a steroid. In some embodiments, the additional therapeutic agent is a corticosteroid. In some embodiments, the steroid is such as triamcinolone, dexamethasone, fluocinolone, cortisone, prednisone, prednisolone, or flumetholone.

In some embodiments, the additional therapeutic agent is an immunosuppressant. In some embodiments, the immunosuppressant is methotrexate or cyclosporin A. In some embodiments, the immunosuppressant is mycophenolate mofetil or mycophenolate sodium.

In some embodiments, the additional therapeutic agent is azelaic acid.

### Pharmaceutical Formulations

Pharmaceutical formulations for topical administration for administration to skin may include solutions, suspensions, foams, ointments, lotions, creams, gels, sprays, liquids, and powders. Conventional pharmaceutical carriers, aqueous, powder or oily bases, thickeners and the like may be necessary or desirable. In some embodiments, the composition is formulated for topical administration by solution, suspension, gel, cream, ointment, lotion, spray, foam, liquid, and powder.

For the treatment of skin disorders such as cutaneous lichen planus (LP), topical drugs, which are able to penetrate the skin barrier and provide limited systemic effects, are of particular importance.

Topical (dermal/intradermal) formulations are typically solutions, suspensions, gels, creams, ointments, lotions, sprays and foams. Preferred topical formulations should be physically and chemically stable, not cause skin irritation, and deliver the active agent at the appropriate layer of the skin in concentrations that would result in therapeutic response, with limited systemic exposure.

In some embodiments, the administration is topical and comprised of formulations with one or more pharmaceutically (e.g., dermatologically) acceptable excipients. Examples of dermatologically acceptable excipients include, but are not limited to, a pH adjusting agents, chelating agents, preservatives, co-solvents, penetration enhancers, humectants, thickening, gelling, viscosity building agents, surfactants, propellants, fragrance, colorants, or any combination or mixture thereof. In some embodiments, the topical formulation is administered locally to the patient (e.g., administered at the site of a lesion).

In some embodiments, the pH-adjusting agent is selected from an acid, an acid salt, a base, a base salt, and a buffer, or any mixture thereof. Exemplary acids include, but are not limited to, lactic acid, acetic acid, citric acid, and benzoic acid, and salts thereof. Exemplary bases include, but are not limited to, trolamine, tromethamine, and salts thereof. Exemplary buffers include, but are not limited to, citrate/citric acid, acetate/acetic acid, edetate/edetic acid, lactate/lactic acid, and the like.

In some embodiments, the chelating agent is a single excipient. In some embodiments, the chelating agent is a mixture of two or more chelating agents. Exemplary chelating agents include, but are not limited to, ethylenediaminetetraacetic acid (EDTA), or a salt thereof. In some embodiments, the chelating agent comprises a mixture of a chelating agent and an antioxidant, wherein the chelating agent and antioxidant prevent, minimize, or reduce oxidative degradation reactions in the composition. Exemplary anti-oxidants include, but are not limited to, butylated hydroxytoluene (BHT), butylated hydroxyanisole (BHA), tocopherol, and propyl gallate.

In some embodiments, the composition comprises one or more preservatives. In some embodiments, the composition comprises a mixture of two or more preservatives. In some embodiments, the composition comprises one to five preservatives. Exemplary preservatives include, but are not limited to, benzyl alcohol, phenonyexthanol, methyl paraben, ethyl paraben, propyl paraben, butyl paraben, and imidazolidinyl urea.

In some embodiments, the composition comprises one or more co-solvents. In some embodiments, the composition comprises a mixture of two or more co-solvents. In some embodiments, the composition comprises one to five co-solvents. Exemplary solvents include, but are not limited to, water, propylene glycol, diethylene glycol monoethyl ether, dimethyl isosorbide, ethyl alcohol, isopropyl alcohol, benzyl alcohol, propanediol, propylene glycol, polyethylene glycols (e.g., polyethylene glycol 200, polyethylene glycol 300, polyethylene glycol 400, and the like). In some embodiments, the solvent is a non-water soluble agent. Exemplary non-water soluble agents include, but are not limited to, diethyl sebacate, diisopropyl adipate, isopropyl myristate, isopropyl palmitate, and medium chain triglycerides.

In some embodiments, the composition comprises one or more penetration enhancers. In some embodiments, the composition comprises a mixture of two or more penetration enhancers. In some embodiments, the composition comprises one to five penetration enhancers. The penetration enhancers can act as both a solvent and a penetration enhancer. Exemplary penetration enhancers include, but are not limited to, fatty acids, fatty acid esters, fatty alcohols, pyrrolidones, sulfoxides, alcohols, diols and polyols, or any mixture thereof. In some embodiments, a co-solvent provided herein is a penetration enhancer.

In some embodiments, the composition comprises one or more thickening, gelling, or viscosity building agents. In some embodiments, the composition comprises a mixture of two or more thickening, gelling, or viscosity building agents. In some embodiments, the composition comprises one to five thickening, gelling, or viscosity building agents. Exemplary thickening, gelling, or viscosity building agents include, but are not limited to, cellulosic derivatives (e.g., hydroxyethylcellulose (HEC), carboxymethylcellulose, hydroxypropylcellulose (HPC), and hydroxypropyl methylcellulose (HPMC), and polyvinylpyrrolidone (PVP).

The surfactant is a compound that lowers the surface tension between two liquids or between a liquid and a solid. Surfactant may be a mixture of two or more surfactants. Exemplary surfactants include, but are not limited to, ethoxylated fatty alcohol ether (e.g., steareth-2, steareth-10, steareth-20, ceteareth-2, ceteareth-10, and the like), PEG esters (e.g., PEG-4 dilaurate, PEG-20 stearate, and the like), Glyceryl esters or derivatives thereof (e.g., glyceryl dioleate, glyceryl stearate, and the like), polymeric ethers (e.g., poloxamer 124, poloxamer 181, poloxamer 182, and the like), sorbitan derivatives (e.g., polysorbate 80, sorbitan monostearate, and the like), fatty alcohols (e.g., cetyl alcohol, stearyl alcohol, cetearyl alcohol, and the like), and emulsifying wax (e.g., emulsifying wax NF, mixtures of mixture of cetearyl alcohol and polysorbate 60, and the like).

Topical (e.g., intradermal) administration provides the advantage of treating the skin disorder locally, minimizing potential adverse events associated with systemic exposure, and allowing an easier discontinuation of the therapy, if necessary. Additionally, some topical dosage forms such as creams, ointments, and gels have the benefit of excipients that may act as emollients or occlusive agents, which can increase patient well-being and compliance during the treatment period. Other dosage routes such as oral, parenteral, and inhalation may lead to supratherapeutic systemic drug levels, increased likelihood of adverse events, drug-drug interactions, and generation of active/toxic metabolites, which may result in treatment discontinuation and inadequate patient compliance.

Topical formulations intended for dermal delivery are typically solutions, suspensions, gels, creams, ointments, lotions, sprays, and foams and can contain one or more conventional carriers as described herein. The formulation composition should be prepared with the goal of delivering the active ingredient to the appropriate layer(s) of the skin, minimizing systemic exposure, and preventing skin irritation. Additionally the pharmaceutical composition should be physically and chemically stable. Depending on the selected dosage form, one or more additional excipients as described herein may be necessary, e.g., pH adjusting agents, chelating agents, preservatives, co-solvents, penetration enhancers, humectants, thickening, gelling, viscosity building agents, surfactants, propellants, fragrances, colorants, or any combination or mixture thereof.

In some embodiments, topical formulations can contain one or more conventional carriers as described herein. In some embodiments, ointments can contain water and one or more hydrophobic carriers selected from, for example, liquid paraffin, polyoxyethylene alkyl ether, propylene glycol, white petrolatum, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The compositions of the invention can further include one or more additional pharmaceutical agents, examples of which are listed hereinabove.

### Cream Formulations

In some embodiments, the cream comprises an oil-in-water emulsion, comprising 1.5% (w/w) on a free base basis of ruxolitinib phosphate.

In some embodiments, the cream is an oil-in-water emulsion as described in US 2015/0250790, which is incorporated herein by reference in its entirety. In particular, Examples 3-6 of US 2015/0250790 (and particularly Tables 3-5 and accompanying text) are incorporated herein by reference.

In some embodiments, the oil component is present in an amount of about 10% to about 40% by weight of the emulsion.

In some embodiments, the oil component is present in an amount of about 10% to about 24% by weight of the emulsion.

In some embodiments, the oil component is present in an amount of about 15% to about 24% by weight of the emulsion.

In some embodiments, the oil component is present in an amount of about 18% to about 24% by weight of the emulsion.

In some embodiments, the oil component comprises one or more substances independently selected from petrolatums, fatty alcohols, mineral oils, triglycerides, and silicone oils.

In some embodiments, the oil component comprises one or more substances independently selected from white petrolatum, cetyl alcohol, stearyl alcohol, light mineral oil, medium chain triglycerides, and dimethicone.

In some embodiments, the oil component comprises an occlusive agent component.

In some embodiments, the occlusive agent component is present in an amount of about 2% to about 15% by weight of the emulsion.

In some embodiments, the occlusive agent component is present in an amount of about 5% to about 10% by weight of the emulsion.

In some embodiments, the occlusive agent component comprises one or more substances selected from fatty acids (e.g., lanolin acid), fatty alcohols (e.g., lanolin alcohol), hydrocarbon oils & waxes (e.g., petrolatum), polyhydric alcohols (e.g., propylene glycol), silicones (e.g., dimethicone), sterols (e.g., cholesterol), vegetable or animal fat (e.g., cocoa butter), vegetable wax (e.g., Carnauba wax), and wax ester (e.g., bees wax).

In some embodiments, the occlusive agent component comprises one or more substances selected from lanolin acid fatty alcohols, lanolin alcohol, petrolatum, propylene glycol, dimethicone, cholesterol, cocoa butter, Carnauba wax, and bees wax.

In some embodiments, the occlusive agent component comprises petrolatum.

In some embodiments, the occlusive agent component comprises white petrolatum.

In some embodiments, the oil component comprises a stiffening agent component.

In some embodiments, the stiffening agent component is present in an amount of about 2% to about 8% by weight of the emulsion.

In some embodiments, the stiffening agent component is present in an amount of about 3% to about 6% by weight of the emulsion.

In some embodiments, the stiffening agent component is present in an amount of about 4% to about 7% by weight of the emulsion.

In some embodiments, the stiffening agent component comprises one or more substances independently selected from fatty alcohols.

In some embodiments, the stiffening agent component comprises one or more substances independently selected from C₁₂₋₂₀ fatty alcohols.

In some embodiments, the stiffening agent component comprises one or more substances independently selected from C₁₆₋₁₈ fatty alcohols.

In some embodiments, the stiffening agent component comprises one or more substances independently selected from cetyl alcohol and stearyl alcohol.

In some embodiments, the oil component comprises an emollient component.

In some embodiments, the emollient component is present in an amount of about 5% to about 15% by weight of the emulsion.

In some embodiments, the emollient component is present in an amount of about 7% to about 13% by weight of the emulsion.

In some embodiments, the emollient component comprises one or more substances independently selected from mineral oils and triglycerides.

In some embodiments, the emollient component comprises one or more substances independently selected from light mineral oil and medium chain triglycerides.

In some embodiments, the emollient component comprises one or more substances independently selected from light mineral oil, medium chain triglycerides, and dimethicone.

In some embodiments, the water is present in an amount of about 35% to about 65% by weight of the emulsion.

In some embodiments, the water is present in an amount of about 40% to about 60% by weight of the emulsion.

In some embodiments, the water is present in an amount of about 45% to about 55% by weight of the emulsion.

In some embodiments, the emulsifier component is present in an amount of about 1% to about 9% by weight of the emulsion.

In some embodiments, the emulsifier component is present in an amount of about 2% to about 6% by weight of the emulsion.

In some embodiments, the emulsifier component is present in an amount of about 3% to about 5% by weight of the emulsion.

In some embodiments, the emulsifier component is present in an amount of about 4% to about 7% by weight of the emulsion.

In some embodiments, the emulsion comprises an emulsifier component and a stiffening agent component, wherein the combined amount of emulsifier component and stiffening agent component is at least about 8% by weight of the emulsion.

In some embodiments, the emulsifier component comprises one or more substances independently selected from glyceryl fatty esters and sorbitan fatty esters.

In some embodiments, the emulsifier component comprises one or more substances independently selected from glyceryl stearate, and polysorbate 20.

In some embodiments, the emulsion further comprises a stabilizing agent component.

In some embodiments, the stabilizing agent component is present in an amount of about 0.05% to about 5% by weight of the emulsion.

In some embodiments, the stabilizing agent component is present in an amount of about 0.1% to about 2% by weight of the emulsion.

In some embodiments, the stabilizing agent component is present in an amount of about 0.3% to about 0.5% by weight of the emulsion.

In some embodiments, the stabilizing agent component comprises one or more substances independently selected from polysaccharides.

In some embodiments, the stabilizing agent component comprises xanthan gum.

In some embodiments, the emulsion further comprises a solvent component.

In some embodiments, the solvent component is present in an amount of about 10% to about 35% by weight of the emulsion.

In some embodiments, the solvent component is present in an amount of about 15% to about 30% by weight of the emulsion.

In some embodiments, the solvent component is present in an amount of about 20% to about 25% by weight of the emulsion.

In some embodiments, the solvent component comprises one or more substances independently selected from alkylene glycols and polyalkylene glycols.

In some embodiments, the solvent component comprises one or more substances independently selected from propylene glycol and polyethylene glycol.

In some embodiments, the emulsion comprises:
from about 35% to about 65% of water by weight of the emulsion;
from about 10% to about 40% of an oil component by weight of the emulsion;
from about 1% to about 9% of an emulsifier component by weight of the emulsion;
from about 10% to about 35% of a solvent component by weight of the emulsion;
from about 0.05% to about 5% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 35% to about 65% of water by weight of the emulsion;
from about 10% to about 24% of an oil component by weight of the emulsion;
from about 1% to about 9% of an emulsifier component by weight of the emulsion;
from about 10% to about 35% of a solvent component by weight of the emulsion;
from about 0.05% to about 5% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 40% to about 60% of water by weight of the emulsion;
from about 15% to about 30% of an oil component by weight of the emulsion;
from about 2% to about 6% of an emulsifier component by weight of the emulsion;
from about 15% to about 30% of a solvent component by weight of the emulsion;
from about 0.1% to about 2% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 40% to about 60% of water by weight of the emulsion;
from about 15% to about 30% of an oil component by weight of the emulsion;
from about 2% to about 6% of an emulsifier component by weight of the emulsion;
from about 15% to about 24% of a solvent component by weight of the emulsion;
from about 0.1% to about 2% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 15% to about 24% of an oil component by weight of the emulsion;
from about 3% to about 5% of an emulsifier component by weight of the emulsion;
from about 20% to about 25% of a solvent component by weight of the emulsion;
from about 0.3% to about 0.5% of a stabilizing agent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 15% to about 24% of an oil component by weight of the emulsion;
from about 4% to about 7% of an emulsifier component by weight of the emulsion;
from about 20% to about 25% of a solvent component by weight of the emulsion;
from about 0.3% to about 0.5% of a stabilizing agent component by weight of the emulsion; and
from 0.5% to 1.5% of ruxolitinib, or a pharmaceutically acceptable salt thereof, by weight of the emulsion on a free base basis.

In some embodiments:
the oil component comprises one or more substances independently selected from petrolatums, fatty alcohols, mineral oils, triglycerides, and dimethicones;
the emulsifier component comprises one or more substances independently selected from glyceryl fatty esters and sorbitan fatty esters;
the solvent component comprises one or more substances independently selected from alkylene glycols and polyalkylene glycols; and
the stabilizing agent component comprises one or more substances independently selected from polysaccharides.

In some embodiments:
the oil component comprises one or more substances independently selected from white petrolatum, cetyl alcohol, stearyl alcohol, light mineral oil, medium chain triglycerides, and dimethicone;
the emulsifier component comprises one or more substances independently selected from glyceryl stearate and polysorbate 20;
the solvent component comprises one or more substances independently selected from propylene glycol and polyethylene glycol; and
the stabilizing agent component comprises xanthan gum.

In some embodiments, the emulsion comprises:
from about 35% to about 65% of water by weight of the emulsion;
from about 2% to about 15% of an occlusive agent component by weight of the emulsion;
from about 2% to about 8% of a stiffening agent component by weight of the emulsion;
from about 5% to about 15% of an emollient component by weight of the emulsion;
from about 1% to about 9% of an emulsifier component by weight of the emulsion;
from about 0.05% to about 5% of a stabilizing agent component by weight of the emulsion;
from about 10% to about 35% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 40% to about 60% of water by weight of the emulsion;
from about 5% to about 10% of an occlusive agent component by weight of the emulsion;
from about 2% to about 8% of a stiffening agent component by weight of the emulsion;
from about 7% to about 12% of an emollient component by weight of the emulsion;
from about 2% to about 6% of an emulsifier component by weight of the emulsion;
from about 0.1% to about 2% of a stabilizing agent by weight of the emulsion;
from about 15% to about 30% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 5% to about 10% of an occlusive agent component by weight of the emulsion;
from about 3% to about 6% of a stiffening agent component by weight of the emulsion;
from about 7% to about 13% of an emollient component by weight of the emulsion;
from about 3% to about 5% of an emulsifier component by weight of the emulsion;
from about 0.3% to about 0.5% of a stabilizing agent component by weight of the emulsion;
from about 20% to about 25% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
from about 5% to about 10% of an occlusive agent component by weight of the emulsion;
from about 4% to about 7% of a stiffening agent component by weight of the emulsion;
from about 7% to about 13% of an emollient component by weight of the emulsion;
from about 4% to about 7% of an emulsifier component by weight of the emulsion;
from about 0.3% to about 0.5% of a stabilizing agent component by weight of the emulsion;
from about 20% to about 25% of a solvent component by weight of the emulsion; and
from 0.5% to 1.5% of ruxolitinib, or a pharmaceutically acceptable salt thereof, by weight of the emulsion on a free base basis.

In some embodiments, the emulsion comprises:
from about 45% to about 55% of water by weight of the emulsion;
about 7% of an occlusive agent component by weight of the emulsion;
from about 4.5% to about 5% of a stiffening agent component by weight of the emulsion;
about 10% of an emollient component by weight of the emulsion;
from about 4% to about 4.5% of an emulsifier component by weight of the emulsion;
about 0.4% of a stabilizing agent component by weight of the emulsion;
about 22% of a solvent component by weight of the emulsion; and
from 0.5% to 1.5% of ruxolitinib, or a pharmaceutically acceptable salt thereof, by weight of the emulsion on a free base basis.

In some embodiments, the ruxolitinib, or pharmaceutically acceptable salt thereof, is present as ruxolitinib phosphate.

In some embodiments, the emulsion comprises 1.5% of ruxolitinib, or a pharmaceutically acceptable salt thereof, by weight of the emulsion.

In some embodiments, the emulsion comprises 1.5% of ruxolitinib phosphate by weight of the emulsion.

In some embodiments, the emulsion comprises 0.75% of ruxolitinib, or a pharmaceutically acceptable salt thereof, by weight of the emulsion.

In some embodiments, the emulsion comprises 0.75% of ruxolitinib phosphate by weight of the emulsion.

In some embodiments, the combined amount of the stiffening agent component and the emulsifier component is at least about 8% by weight of the emulsion.

In some embodiments:
the occlusive agent component comprises a petrolatum;
the stiffening agent component comprises one or more substances independently selected from one or more fatty alcohols;
the emollient component comprises one or more substances independently selected from mineral oils and triglycerides;
the emulsifier component comprises one or more substances independently selected from glyceryl fatty esters and sorbitan fatty esters;
the stabilizing agent component comprises one or more substances independently selected from polysaccharides; and
the solvent component comprises one or more substances independently selected from alkylene glycols and polyalkylene glycols.

In some embodiments:
the occlusive agent component comprises white petrolatum;
the stiffening agent component comprises one or more substances independently selected from cetyl alcohol and stearyl alcohol;
the emollient component comprises one or more substances independently selected from light mineral oil, medium chain triglycerides, and dimethicone;
the emulsifier component comprises one or more substances independently selected from glyceryl stearate and polysorbate 20;
the stabilizing agent component comprises xanthan gum; and
the solvent component comprises one or more substances independently selected from propylene glycol and polyethylene glycol.

In some embodiments, the emulsion further comprises an antimicrobial preservative component.

In some embodiments, the antimicrobial preservative component is present in an amount of about 0.05% to about 3% by weight of the emulsion.

In some embodiments, the antimicrobial preservative component is present in an amount of about 0.1% to about 1% by weight of the emulsion.

In some embodiments, the antimicrobial preservative component comprises one or more substances independently selected from alkyl parabens and phenoxyethanol.

In some embodiments, the antimicrobial preservative component comprises one or more substances independently selected from methyl paraben, propyl paraben, and phenoxyethanol.

In some embodiments, the emulsion further comprises a chelating agent component.

In some embodiments, the chelating agent component comprises edetate disodium.

Ruxolitinib can be prepared as described in U.S. Pat. No. 7,598,257 and U.S. Patent Publ. No. 2009/0181959, each of which is incorporated herein by reference in its entirety. The 1:1 phosphate salt of ruxolitinib can be prepared as described in U.S. Patent Publ. No. 2008/0312259, which is incorporated herein by reference in its entirety.

As will be appreciated, some components of the cream (emulsion) described herein can possess multiple functions. For example, a given substance may act as both an emulsifying agent component and a stabilizing agent. In some such cases, the function of a given component can be considered singular, even though its properties may allow multiple functionality. In some embodiments, each component of the formulation comprises a different substance or mixture of substances.

### Kits

The present application also includes pharmaceutical kits useful, for example, in the treatment and/or prevention of lichen planus (LP), which include one or more containers containing a pharmaceutical composition comprising a therapeutically effective amount of ruxolitinib, or a pharmaceutically acceptable salt thereof, as described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters, which can be changed or modified to yield essentially the same results.

All statistical analysis of in vitro experiments were performed with GraphPad prism software (version 7) using Kruskal-Wallis-Test and Mann-Whitney U test. Gene expression was analyzed with Partek Flow genomic analysis software and Subio Platform software v1.22.5266 using Welch's t-test. Confidence intervals were determined at 95%. P<0.05 was considered to be “significant” (*), p<0.01 to be “highly significant” (**). KEGG pathways were mapped to differentially expressed genes using DAVID v6.8 (Database for Annotation, Visualization and Integrated Discovery).

### Example A: Preparation of Oil-In-Water Cream Formulations of Ruxolitinib (INCB018424) Phosphate

An oil-in-water cream formulation was prepared for 1:1 ruxolitinib phosphoric acid salt at 1.5% by weight of the formulation (free base equivalent). All excipients used in the formulation were compendial grade (i.e., USP/NF or BP) or are approved for use in topical products. The quantitative formulae for representative 400 kg batches of the cream formulation at 0.5, 1.0 and 1.5% are also provided in Tables A.

The oil-in-water cream formulations were synthesized according to the following procedure. Some batches were subject to minor changes associated with scale-up, such as the size of mixing vessels and mixers. Generally, overhead mixer with high and low shear mixing blades are suitable for the process.

Procedure
1. A paraben phase was prepared by mixing methyl and propyl parabens with a portion of the propylene glycol (see % in Table A).
2. Next, a xanthan gum phase was prepared by mixing xanthan gum with propylene glycol (see % in Table A).
3. An oil phase was then prepared by mixing light mineral oil, glyceryl stearate, polysorbate 20, white petrolatum, cetyl alcohol, stearyl alcohol, dimethicone and medium chain triglycerides. The phase is heated to 70-80° C. to melt and form a uniform mixture.
4. The aqueous phase was next prepared by mixing purified water, polyethylene glycol, and disodium EDTA. The phase is heated to 70-80° C.
5. The aqueous phase of step 4, paraben phase of step 1, and Example 2 (phosphate salt of API) were combined to form a mixture.
6. The xanthan gum phase from step 2 was then added to the mixture from step 5.
7. The oil phase from step 3 was then combined under high shear mixing with the mixture from step 6 to form an emulsion.
8. Phenoxyethanol was then added to the emulsion from step 7. Mixing was continued, and then the product was cooled under low shear mixing.

More consistent batches at larger scales (e.g., 140 kg) could be obtained by adding ruxolitinib phosphate gradually to the aqueous phase and then combining with the other phases. Similarly, more consistent batches could be obtained by slower cooling (e.g., by using room temperature water in the outer jacket of the reactor, rather than lower temperature water.

The batches were tested for stability at 25° C. and found to be stable up to 24 months with a pH consistent with the pH range described supra (see Table 7, 9, 11, 12, 13, 15, 17, and 19 in U.S. Patent Publ. No. 2015/0250790, which is incorporated herein by reference in its entirety). The viscosity of the cream formulations (e.g., containing 0.75% or 1.5% ruxolitinib phosphate on a free base basis) had a viscosity of ≥17,000 cPs on release and a shelf-life viscosity of ≥10,000 cPs. The pH of the batch in Table A was 2.8 to 3.6.

TABLE A

| Ingredient | Kilograms | Percentage (w/w) |
|---|---|---|
| Ruxolitinib phosphate | 7.92 (phosphate | 1.98 (phophate |
| | salt)/ | salt)/ |
| | 6.0 (free base) | 1.5 (free base) |
| Propylene Glycol USP | 40.0 | 10.00 |
| Methyl Paraben NF | 0.4 | 0.10 |
| Propyl Paraben NF | 0.2 | 0.05 |
| Propylene Glycol USP | 20.0 | 5.00 |
| Xanthan Gum NF | 1.6 | 0.40 |
| Light Mineral Oil NF | 16.0 | 4.00 |
| Glyceryl Stearate SE | 12.0 | 3.00 |
| Polysorbate 20 NF | 5.0 | 1.25 |
| White Petrolatum USP | 28.0 | 7.00 |
| Cetyl alcohol NF | 12.0 | 3.00 |
| Stearyl alcohol NF | 7.0 | 1.75 |
| Dimethicone 360 NF | 4.0 | 1.00 |
| Medium Chain Triglycerides NF | 20.0 | 5.00 |
| Purified Water USP (approximate) | 195.5 | 48.9 |
| Edetate Disodium USP | 0.2 | 0.05 |
| Polyethylene Glycol USP | 28.0 | 7.00 |
| Phenoxyethanol BP | 2.0 | 0.5 |
| Total (approximate) | 400.0 | 100 |

### Example 1. Phase II Study Regarding Treatment of Cutaneous Lichen Planus with Ruxolitinib

NCT03697460 was a single center, exploratory, open-label, single-arm design study of 12 patients. Patients who were non-responders, to physician choice standard of care, underwent a washout period and were enrolled in the study.

Multiple safety studies have been conducted with ruxolitinib (INCB018424) phosphate cream and a dose of 1.5% cream BID on up to 20% BSA is deemed safe. Within each cohort, the maximum reduction from baseline in the total psoriasis lesion assessment scores for the treated lesions was observed at the end of treatment (Day 28). Therefore, a single center, exploratory, open-label, single-arm design study of 12 patients were conducted as described herein. Treatment naïve and treatment refractory patients with LP were treated with 1.5% ruxolitinib (INCB018424) phosphate cream. The ruxolitinib in the ruxolitinib cream was present as ruxolitinib phosphate with the percentages as % w/w on a free base basis. The ruxolitinib cream formulations were oil-in-water cream formulations prepared as described in Example A (see also U.S. Patent Publ. No. 2015/0250790), which is incorporated herein by reference in its entirety.

Individuals with LP up to 20%, such as between 2% and 20%, of the BSA were selected. The selected individuals had a minimum of 4 lesions, preferably 10 lesions or more, which were at least 5 mm in diameter. 10 lesions were ideal as 2 lesions were index lesions and 8 or more lesions were selected from as responsive or non-responsive at 4 weeks. All lesions except for the index lesion were treated and all lesions were annotated, photographed, and scored. The rationale for a minimum of 8 lesions is based upon the assumption of a 50% response rate at 2 weeks. With 8 lesions, there would be a less than 1% chance that all 8 lesions would respond. Ideally, the 2 index lesions would be 10 mm in diameter but this was not required. Prior treatment were allowed; however, a washout period of 2 weeks for topical and 4 weeks for systemic agents was required. At the washout period, individuals underwent evaluation with a PhCLPGA, BSA calculation, and had the 2 largest, representative lesions selected for evaluation using a modified CAILS score lesion A-treatment index lesion and B-index control lesion. The determination of which lesion receives treatment and which was a control were determined randomly. The additional 8 lesions were photographed, measured, scored, and recorded with CAILS scores as well. The later calculations were used for the determination of responsive and non-responsive lesions. Additional Pruritus measures using the NRS and Skindex-16 were collected at that time.

In cases where some or all of the affected body regions contain such extensive LP disease that make counting individual LP lesions unfeasible, the lesion count in these areas were estimated. The palmar surface of the hand equates to approximately 1% BSA. To estimate the lesion count in a body region of extensive disease, the number of lesions counted within an area equivalent to area the palmar surface of the hand within that extensively diseased body region was multiplied by the representative BSA of that body region to determine the estimated lesion count contained in that body region. Representative BSAs of body regions are defined as follows: head (7%), neck (2%), anterior trunk (13%), arms (8%), forearms (6%), hands (5%), posterior trunk (13%), buttocks (5%), thighs (19%), legs (14%), feet (7%) and groin (1%).

Individuals were initially treated on all lesions of LP BID and were evaluated weekly and assessed by PhCLPGA, BSA, lesion count; CAILS, Pruritus NRS, and Skindex-16 between weeks 0-4 (see Appendix). At week 4 were the primary endpoint; however, therapy were continued for an additional 4 weeks. Therapy were stopped and the individuals were evaluated at week 8 and assessed by PhCLPGA, BSA, lesion count, CAILS, Pruritus NRS, and Skindex-16. Laboratory and safety monitoring will occur at weeks 1, 2, 3, 4, and 8, and 12. 3D Photographs were taken at weeks 0, 2, 4, 8, and 12. Individual lesions were circled at that time and the exact volume of each lesion were measured. Up close photos were taken of the disease and normal tissues attained at weeks 4, & 12.

Responsive and non-responsive lesions were determined by the change in CAILS between week 0 and 4. Responsive would require a 50% reduction in CAILS and non-responsive could have progression, no change, or up to a 50% reduction in CAILS.

Subjects who met all of the following key inclusion criteria could be included in the study: (i) men and women aged 18 of age at the time of screening; (ii) have clinical and histological features of LP; (iii) LP must involve between 2 and 20% of the BSA; (iv) have a minimum of 4 lesions, preferably 10 lesions of LP; (v) have treatment naive cutaneous LP or treatment refractory disease, as defined by failure of at least one established treatment for LP; and (vi) failed at least one of the prior therapies including but not limiting to topical treatment, systemic immunosuppressant, oral metronidazole, oral sulfasalazine, and oral retinoid.

Subjects who met any of the following key exclusion criteria were excluded from the study: (i) on excluded therapies, not on a stable dose of a therapy, or incompletely washed out for a therapy (Table-1); (ii) known hypersensitivity to any component of INCB018424 PHOSPHATE CREAM; (iii) variants of LP deemed by the investigators to be inappropriate for INCB018424 PHOSPHATE CREAM including by not limited to: erosive LP, intertriginous LP, oral LP, facial LP, drug-induced LP, and vaginal LP; (iv) LP involving greater than 20% BSA; (v) pregnant or nursing (lactating) women (pregnancy was defined as the state of a female after conception and until the termination of gestation, confirmed by a positive human chorionic gonadotropin (hCG) laboratory test); (vi) Women of childbearing potential [Post-menopausal or not of child-bearing potential was defined by: 1 year of natural (spontaneous) amenorrhea or Surgical bilateral oophorectomy (with or without hysterectomy), total hysterectomy or tubal ligation at least 6 weeks ago. Oophorectomy alone must be confirmed by follow up hormone level assessment to be considered not of child-bearing potential, defined as all women physiologically capable of becoming pregnant, unless they are using basic methods of contraception which includes: total abstinence (Periodic abstinence and withdrawal are not acceptable methods of contraception); female sterilization (bilateral oophorectomy with or without hysterectomy), total hysterectomy, or tubal ligation at least 6 weeks before taking study treatment (Oophorectomy alone requires follow up hormone level assessment for fertility); male sterilization (at least 6 months prior to screening) (The vasectomized male partner should be the sole partner for that subject); barrier methods of contraception: condom or occlusive cap; use of oral, injected or implanted hormonal methods of contraception or other forms or hormonal contraception that have complete efficacy (failure<1%) (The dose of the contraceptive should be stable for 3 months); (vii) active ongoing inflammatory diseases of the skin other than LP that might confound the evaluation of the benefit of INCB018424 PHOSPHATE CREAM; (viii) underlying condition (including, but not limited to metabolic, hematologic, renal, hepatic, pulmonary, neurologic, endocrine, cardiac, infectious or gastrointestinal conditions) which, in the opinion of the investigator, significantly immunocompromised the subject and/or places the subject at unacceptable risk for receiving an immunomodulatory therapy; (ix) active systemic infections during the 2 weeks prior to randomization (common cold viruses not included) or any infection that reoccurs on a regular basis; and (x) current severe progressive or uncontrolled disease which the investigator renders the subject unsuitable for the trial or puts the subject at increased risk.

TABLE 1

| | Washout period |
|---|---|
| | (before |
| Prohibited treatments^{†, ‡} | Randomization Visit) |
| Any concomitant oral or topical JAK inhibitor | Prohibited |
| Any biologic drug | Stable dose for 3 |
| | months |
| Immunomodulation treatments for LP^{§} | 4 weeks |
| [e.g., methotrexate, cyclosporine A, corticosteroids (oral, i.v., | |
| intramuscular, s.c., intra-articular, transdermal), | |
| mycophenolate mofetil, azathioprine] | |
| Topical treatment that was likely to impact signs and | 2 weeks |
| symptoms of LP (e.g., pimecrolimus, tacrolimus) | |
| Non-immunosuppressive agents (tetracycline antibiotics & | 2 weeks |
| niacinamide) | |
| Prohibited regimen of Topical Corticosteroids (TCS) | |
| TCS on any location on body (including face, scalp and/or | 2 weeks |
| genitoanal area) | |

The primary endpoint of the study is the change from baseline in modified Clinical Assessment Scale of Severity for Index Lesion Signs and Symptoms (mCAILS) score from baseline to Week 4 in subjects treated with 1.5% ruxolitinib phosphate cream BID compared with subjects treated with vehicle cream BID; the change of total lesion count from baseline to Week 4 in subjects treated with 1.5% ruxolitinib phosphate cream BID compared with subjects treated with vehicle cream BID.

Secondary endpoint of the study is the change in Pruritus NRS, (week 0 to week 4, week 0 to week 8, weeks 4 to 8, weeks 8 to 12); change in Skindex-16 (week 0 to week 4, weeks 4 to 8, weeks 8 to 12); change in Physician Global Cutaneous Lichen Planus Assessment (PhCLPGA) (week 0 to week 4, week 0 to week 8, weeks 4 to 8, weeks 8 to 12); change in BSA (week 0 to week 4, week 0 to week 8, weeks 4 to 8, weeks 8 to 12); change in modified CAILS (week 0 to week 8, weeks 4 to 8, weeks 8 to 12).

Exploratory outcome measures is to predict responses and examine the pharmacodynamics of treatment through the identification of unique biomarkers and transcriptomic changes of LP at week 0 and utilizing RNA sequencing on responsive and non-responsive tissue at week 4 To correlate these biomarkers with measures of global response: modified CAILS, PhCLPGA, BSA, and lesion count.

### Results

Summary: Patients (83.3% female) had a mean age of 61.1 years. Total lesion count decreased by a median of 50 lesions (interquartile range [IQR] 25, 723; p<0.001). mCAILS scores decreased by a mean difference of 7.6 (SD 8.8, p=0.016) between index treatment and control lesions. Ten index treatment lesions (83.3%) achieved treatment response as defined by ≥50% reduction in mCAILS score compared to 4/12 (33.3%) of controls (p=0.077). Excluding 4 patients who treated their index control lesion, 7 (87.5%) treatment compared to 1 (12.5%) control lesions responded (p=0.04). Affected BSA decreased from an average of 6.1% to 0.9% (p=0.004). All patients were responsive by PGA: 2/12 were 100% clear; 5/12 had ≥90% improvement; 4/12 had marked (≥75-90%) improvement; 1/12 had moderate (≥50-75%) improvement.

Demographics and baseline clinical characteristics for the enrolled patients are shown in Table 2.

TABLE 2

| | Overall (N = 12) | |
|---|---|---|
| Age (in years) | | |
| Mean (SD) | 61.1 | (14.0) |
| Median | 63.0 | |
| Q1, Q3 | 53.2, 69.8 | |
| Range | 34.0-81.0 | |
| Sex at Birth | | |
| Female | 10 | (83.3%) |
| Male | 2 | (16.7%) |
| Ethnicity | | |
| Non Hispanic or Latino | 10 | (83.3%) |
| Hispanic or Latino | 1 | (8.3%) |
| Not reported | 1 | (8.3%) |
| Race | | |
| Caucasian | 11 | (91.7%) |
| Other | 1 | (8.3%) |
| Is subject of Child Bearing Potential? | | |
| N-Miss | 2 | |
| Able to Bear Children | 2 | (20.0%) |
| Premenarche | 1 | (10.0%) |
| Post-Menopausal | 7 | (70.0%) |
| Sterile | 0 | (0.0%) |
| Serum Pregnancy Test collected? | | |
| No | 9 | (75.0%) |
| Yes | 3 | (25.0%) |
| Serum Pregnancy Test | | |
| N-Miss | 9 | |
| Negative | 3 | (100.0%) |
| Positive | 0 | (0.0%) |
| Lesion Randomization | | |
| Largest lesion - Index Treatment | 6 | (50.0%) |
| Largest lesion - Index Control | 6 | (50.0%) |
| Prior Therapies: Topical | | |
| Yes | 12 | (100.0%) |
| Prior Therapies: Oral Immunosuppressant | | |
| No | 8 | (66.7%) |
| Yes | 4 | (33.3%) |
| Prior Therapies: Oral Non-immunosuppressant | | |
| No | 11 | (91.7%) |
| Yes | 1 | (8.3%) |
| Number of prior therapies taken | | |
| 1 | 7 | (58.3%) |
| 2 | 2 | (16.7%) |
| 3 | 3 | (25.0%) |
| Was the chest X-RAY normal? | | |
| No | 0 | (0.0%) |
| Yes | 12 | (100.0%) |
| Quantiferon Gold result | | |
| Negative | 12 | (100.0%) |
| Positive | 0 | (0.0%) |
| Hepatitis B result | | |
| Negative | 12 | (100.0%) |
| Positive | 0 | (0.0%) |
| Hepatitis C result | | |
| Negative | 12 | (100.0%) |
| Positive | 0 | (0.0%) |
| HIV result | | |
| Negative | 12 | (100.0%) |
| Positive | 0 | (0.0%) |
| Coccidioidomycosis result | | |
| Negative | 12 | (100.0%) |
| Positive | 0 | (0.0%) |

As shown in Table 3, patients achieved significant reductions in the total body lesion count over 12 week of the administering. For example, the median patient achieved lesion reductions in the total body lesion court: 53-lesion reduction from baseline (day 0) to week 4; 69 lesion reduction from baseline (day 0) to week 8; 16 lesion reduction from baseline week 4 to week 8 and 3 lesion reduction from week 8 to week 12. Based on the mean scores, patients also achieved significant reductions in the total body lesion count over 12 week period: 63 lesion reduction from baseline (day 0) to week 4; 84 lesion reduction from baseline (day 0) to week 8; 21 lesion reduction from baseline week 4 to week 8.

TABLE 3

| Total body | Day 0 | Week 1 | Week 2 | Week 3 | Week 4 | Week 8 | Week 12 |
|---|---|---|---|---|---|---|---|
| lesion count | (N = 12) | (N = 11) | (N = 12) | (N = 12) | (N = 12) | (N = 10) | (N = 6) |
| Mean (SD) | 86.6 | 48.6 | 61.6 | 33.3 | 22.3 | 2.0 | 6.1 |
| | (150.8) | (99.1) | (132.4) | (74.0) | (68.3) | (4.5) | (13.9) |
| Median | 74.5 | 53 | 37.5 | 33 | 21 | 5 | 2 |

SD: Standard Deviation

Modified CAILS scores for control lesion and treatment lesion of the patients are shown in Table 4 and Table 5 respectively. Comparing mean modified CAILS score for treatment lesion to that of control lesion, the patients achieved reductions in modified CAMS score over 12 week of the administering: 11-point reduction from baseline (day 0) to week 4; 11-point reduction from baseline (day 0) to week 8; and further 2-point reduction from week 8 to week 12. In addition, the modified CAILS scores sum excluding control lesion is shown in Table 6, which also shows that the patients achieved a reduction in modified CAILS score over 12 week period.

TABLE 4

| Modified CAILS | | | | | | | |
|---|---|---|---|---|---|---|---|
| score for control | Day 0 | Week 1 | Week 2 | Week 3 | Week 4 | Week 8 | Week 12 |
| lesion | (N = 12) | (N = 11) | (N = 12) | (N = 12) | (N = 12) | (N = 10) | (N = 6) |
| N-Miss | 0 | 0 | 0 | 0 | 0 | 1 | 1 |
| Mean (SD) | 11.7 | 12.1 | 10.2 | 9.1 | 8.2 | 3.8 | 6.2 |
| | (4.8) | (5.1) | (5.0) | (6.9) | (7.1) | (6.4) | (9.1) |
| Median | 12 | 13 | 8.5 | 8.5 | 8 | 0 | 0 |

SD: Standard Deviation

TABLE 5

| Modified CAILS | | | | | | | |
|---|---|---|---|---|---|---|---|
| score for | Day 0 | Week 1 | Week 2 | Week 3 | Week 4 | Week 8 | Week 12 |
| treatment lesion | (N = 12) | (N = 11) | (N = 12) | (N = 12) | (N = 12) | (N = 10) | (N = 6) |
| N-Miss | 0 | 0 | 0 | 0 | 0 | 1 | 1 |
| Mean (SD) | 14.7 | 9.8 | 7.8 | 6.3 | 3.7 | 3.8 | 1.8 |
| | (5.6) | (6.5) | (5.2) | (4.8) | (4.2) | (4.9) | (4.0) |
| Median | 14.5 | 8 | 7.5 | 6.5 | 2.5 | 0 | 0 |

SD: Standard Deviation

TABLE 6

| Modified CAILS | | | | | | | |
|---|---|---|---|---|---|---|---|
| score sum | | | | | | | |
| excluding control | Day 0 | Week 1 | Week 2 | Week 3 | Week 4 | Week 8 | Week 12 |
| lesion | (N = 12) | (N = 11) | (N = 12) | (N = 12) | (N = 12) | (N = 10) | (N = 6) |
| N-Miss | 0 | 0 | 0 | 0 | 0 | 1 | 1 |
| Mean (SD) | 101.5 | 60.7 | 43.5 | 34.5 | 23.2 | 12.4 | 11.2 |
| | (37.9) | (38.7) | (30.8) | (33.6) | (31.1) | (16.7) | (17.2) |
| Median | 104 | 61 | 42.5 | 22.5 | 10.5 | 8 | 0 |

SD: Standard Deviation

As shown in Table 7, patients achieved significant improvements in the Physician Global Cutaneous Lichen Planus Assessment (PhCLPGA) score over 12 week of the administering. For example, from baseline (day 0) to week 4, 2 out of 12 patients achieved PhCLPGA score of 0 (clear; no evidence of disease); 5 out of 12 patients achieved PhCLPGA score of 1 (almost clear; very significant clearance); 4 out of 12 patients achieved PhCLPGA score of 2 (marked improvement; significant improvement); and 1 out of 12 patients achieved PhCLPGA score of 3 (moderate improvement; intermediate between slight and marked). From baseline (day 0) to week 8; 3 out of 12 patients achieved PhCLPGA score of 0 (clear; no evidence of disease); 6 out of 12 patients achieved PhCLPGA score of 1 (almost clear; very significant clearance); and 1 out of 12 patients achieved PhCLPGA score of 2 (marked improvement; significant improvement). From baseline week 4 to week 8, one more patient achieved PhCLPGA score of 0 or 1.

TABLE 7

| PhCLPGA | Day 0 | Week 1 | Week 2 | Week 3 | Week 4 | Week 8 | Week 12 |
|---|---|---|---|---|---|---|---|
| score | (N = 12) | (N = 11) | (N = 12) | (N = 12) | (N = 12) | (N = 10) | (N = 6) |
| N-Miss | 12 | 0 | 0 | 0 | 0 | 0 | 0 |
| 0 | 0 | 0 | 0 | 1 | 2 | 3 | 3 |
| | | (0.0%) | (0.0%) | (8.3%) | (16.7%) | (30.0%) | (50.0%) |
| 1 | 0 | 1 | 2 | 3 | 5 | 6 | 2 |
| | | (9.1%) | (16.7%) | (25.0%) | (41.7%) | (60.0%) | (33.3%) |
| 2 | 0 | 1 | 3 | 4 | 4 | 1 | 0 |
| | | (9.1%) | (25.0%) | (33.3%) | (33.3%) | (10.0%) | (0.0%) |
| 3 | 0 | 2 | 5 | 4 | 1 | 0 | 0 |
| | | (18.2%) | (41.7%) | (33.3%) | (8.3%) | (0.0%) | (0.0%) |
| 4 | 0 | 6 | 1 | 0 | 0 | 0 | 0 |
| | | (54.5%) | (8.3%) | (0.0%) | (0.0%) | (0.0%) | (0.0%) |
| 5 | 0 | 1 | 1 | 0 | 0 | 0 | 1 |
| | | (9.1%) | (8.3%) | (0.0%) | (0.0%) | (0.0%) | (16.7%) |

As shown in Table 8, based on the mean scores, patients achieved reductions in the Body Surface Area (BSA) score of LP over the 12 weeks of the administering: 5.2-point reduction from baseline (day 0) to week 4; 6-point reduction from baseline (day 0) to week 8; and further 0.8-point reduction from week 4 to week 8.

TABLE 8

| BSA Score | Day 0 | Week 1 | Week 2 | Week 3 | Week 4 | Week 8 | Week 12 |
|---|---|---|---|---|---|---|---|
| (%) | (N = 12) | (N = 11) | (N = 12) | (N = 12) | (N = 12) | (N = 10) | (N = 6) |
| Mean | 6.1 | 3.4 | 3.0 | 1.7 | 0.9 | 0.1 | 0.9 |
| (SD) | (7.9) | (6.5) | (5.2) | (2.6) | (1.9) | (0.2) | (2.0) |
| Median | 4 | 1 | 0.9 | 0.6 | 0.2 | 0.1 | 0.1 |

SD: Standard Deviation

As shown in Table 9, based on the mean scores, patients achieved reductions in the SKindex-16 score over the 12 weeks of the administering: 36.4-point reduction from baseline (day 0) to week 4; 42.2-point reduction from baseline (day 0) to week 8; and further 5.8-point reduction from week 4 to week 8.

TABLE 9

| | Day 0 | Week 1 | Week 2 | Week 3 | Week 4 | Week 8 | Week 12 |
|---|---|---|---|---|---|---|---|
| Skindex-16 | (N = 12) | (N = 11) | (N = 12) | (N = 12) | (N = 12) | (N = 10) | (N = 6) |
| Mean (SD) | 56.2 | 32.6 | 26.3 | 21.8 | 19.8 | 14.0 | 20.0 |
| | (21.6) | (19.5) | (21.4) | (17.6) | (18.6) | (13.4) | (20.0) |
| Median | 56.5 | 37 | 26.5 | 26 | 15 | 13 | 18.5 |

SD: Standard Deviation

As shown in Table 9, based on the mean scores, patients achieved reductions in the pruritus Numerical Rating Scale (NRS) score over the 12 weeks of the administering: 4.5-point reduction from baseline (day 0) to week 4; 4.7-point reduction from baseline (day 0) to week 8; and further 0.2-point reduction from week 4 to week 8.

TABLE 10

| Pruritus | Day 0 | Week 1 | Week 2 | Week 3 | Week 4 | Week 8 | Week 12 |
|---|---|---|---|---|---|---|---|
| NRS | (N = 12) | (N = 11) | (N = 12) | (N = 12) | (N = 12) | (N = 10) | (N = 6) |
| N-Miss | 1 | 0 | 0 | 0 | 0 | 0 | 0 |
| Mean (SD) | 5.8 | 2.7 | 2.0 | 1.3 | 1.3 | 1.1 | 2.0 |
| | (3.5) | (2.7) | (2.2) | (2.1) | (2.0) | (2.8) | (3.5) |
| Median | 7 | 1 | 1 | 0 | 0 | 0 | 0.5 |

SD: Standard Deviation

Although there are total 14 adverse events, none of the adverse events are serious adverse events and only one event at week 1 (“Abnormal taste”) was related to study treatment and others were all not related to study treatment.

Various modifications of the invention, in addition to those described herein, will be apparent to those skilled in the art from the foregoing description. Such modifications are also intended to fall within the scope of the appended claims. Each reference cited in the present disclosure, including all patent, patent applications, and publications, is incorporated herein by reference in its entirety.

1. A method of treating cutaneous lichen planus in a human patient in need thereof, comprising administering to an affected area of said human patient, a topical formulation two times per day, wherein said topical formulation comprises 1.5% (w/w) on a free base basis of ruxolitinib, or a pharmaceutically acceptable salt thereof.

2. The method of claim 1, wherein the patient achieves a reduction in total lesion count from baseline.

3. The method of claim 1, wherein the patient achieves an improvement in Index Treatment and Control Lesion by modified Clinical Assessment Scale of Severity for Index Lesion Signs and Symptoms (CAILS) score from baseline.

4. The method of claim 1, wherein the ruxolitinib, or a pharmaceutically acceptable salt, is ruxolitinib phosphate.

5. The method of claim 1, wherein the patient is a man or woman aged≥18 years.

6. The method of claim 1, wherein the patient has clinical and histological features of lichen planus.

7. The method of claim 1, wherein the patient has a Body Surface Area of lichen planus involvement of up to 20% at baseline.

8. The method of claim 1, wherein the patient has a Body Surface Area of lichen planus involvement of 2% to 20% at baseline.

9. The method of claim 1, wherein the patient has a minimum of 4 lesions of lichen planus.

10. (canceled)

11. (canceled)

12. The method of claim 1, wherein the patient has lichen planus for at least 8 months.

13. The method of claim 1, wherein the patient has failed to achieve an adequate response to prior therapy for lichen planus.

14. The method of claim 13, wherein the prior therapy is administration of one of the following:
one or more topical corticosteroids selected from the group of triamcinolone acetonide, clobetasol proprionate, fluocinolone acetonide, betamethasone valerate, betamethasone dipropionate, amcinonide, desoximetasone, fluocinonide, and halcinonide;
topical tacrolimus and pimecrolimus;
systemic immunosuppressant;
oral metronidazole;
oral sulfasalazine; and
oral retinoid.

15. The method of claim 1, wherein the patient achieves a statistically significant reduction in the total lesion count from baseline.

16. (canceled)

17. (canceled)

18. (canceled)

19. (canceled)

20. The method of claim 15, wherein the patient achieves the reduction of the total lesion count from baseline to week 4 of said administering.

21. The method of claim 15, wherein the patient achieves the reduction of the total lesion count from baseline to week 8 of said administering.

22. The method of claim 15, wherein the patient achieves the reduction of the total lesion count from week 4 to week 8 of said administering.

23. The method of claim 15, wherein the patient achieves the reduction of the total lesion count from week 8 to week 12 of said administering.

24. The method of claim 1, wherein the patient achieves a statistically significant reduction in modified Clinical Assessment Scale of Severity for Index Lesion Signs and Symptoms score for treatment lesion from baseline.

25. (canceled)

26. (canceled)

27. (canceled)

28. (canceled)

29. (canceled)

30. (canceled)

31. (canceled)

32. The method of claim 24, wherein the patient achieves the reduction in modified Clinical Assessment Scale of Severity for Index Lesion Signs and Symptoms score for treatment lesion from baseline to week 4 of said administering.

33. The method of claim 24, wherein the patient achieves the reduction in modified Clinical Assessment Scale of Severity for Index Lesion Signs and Symptoms score for treatment lesion from baseline to week 8 of said administering.

34. The method of claim 24, wherein the patient achieves the reduction in modified Clinical Assessment Scale of Severity for Index Lesion Signs and Symptoms score for treatment lesion from week 4 to week 8 of said administering.

35. The method of claim 24, wherein the patient achieves the reduction in modified Clinical Assessment Scale of Severity for Index Lesion Signs and Symptoms score for treatment lesion from week 8 to week 12 of said administering.

36. The method of claim 24, wherein the patient achieves a statistically significant difference between the modified Clinical Assessment Scale of Severity for Index Lesion Signs and Symptoms score reductions for treatment lesion and control lesion.

37. (canceled)

38. (canceled)

39. (canceled)

40. (canceled)

41. (canceled)

42. (canceled)

43. (canceled)

44. The method of claim 1, wherein the patient achieves a statistically significant reduction in the percentage of the Body Surface Area of the cutaneous lichen planus involvement.

45. (canceled)

46. (canceled)

47. (canceled)

48. The method of claim 1, wherein the patient achieves a Physician Global Cutaneous Lichen Planus Assessment of 0 (clear), 1 (almost clear), or 2 (significant improvement).

49. (canceled)

50. (canceled)

51. (canceled)

52. The method of claim 48, wherein the patient achieves Physician Global Cutaneous Lichen Planus Assessment of 0 (clear), 1 (almost clear), or 2 (significant improvement) at week 4 of said administering.

53. The method of claim 48, wherein the patient achieves Physician Global Cutaneous Lichen Planus Assessment of 0 (clear), 1 (almost clear), or 2 (significant improvement) at week 8 of said administering.

54. The method of claim 1, wherein the patient achieves a statistically significant reduction in pruritus Numerical Rating Scale score from baseline.

55. (canceled)

56. (canceled)

57. (canceled)

58. (canceled)

59. The method of claim 54, wherein the patient achieves the reduction in pruritus Numerical Rating Scale score from baseline to week 4 of said administering.

60. The method of claim 54, wherein the patient achieves the reduction in pruritus Numerical Rating Scale score from baseline to week 8 of said administering.

61. The method of claim 54, wherein the patient achieves the reduction in pruritus Numerical Rating Scale score from week 4 to week 8 of said administering.

62. The method of claim 54, wherein the patient achieves the reduction in pruritus Numerical Rating Scale score from week 8 to week 12 of said administering.

63. The method of claim 1, wherein the patient achieves a statistically significant reduction in the Skindex-16 score.

64. (canceled)

65. (canceled)

66. (canceled)

67. (canceled)

68. The method of claim 63, wherein the patient achieves the reduction in Skindex-16 score from baseline to week 4 of said administering.

69. The method of claim 63, wherein the patient achieves the reduction in Skindex-16 score from baseline to week 8 of said administering.

70. The method of claim 63, wherein the patient achieves the reduction in Skindex-16 score from week 4 to week 8 of said administering.

71. The method of claim 63, wherein the patient achieves the reduction in Skindex-16 score from week 8 to week 12 of said administering.

72. The method of claim 1, wherein said administering is maintained for at least 4 week.

73. The method of claim 1, wherein said administering is maintained for at least 8 weeks.

74. The method of claim 1, wherein said administering is maintained for at least 12 weeks.

75. The method of claim 1, wherein the topical formulation is a cream.

76. The method of claim 75, wherein the cream formulation is an oil-in-water emulsion.

77. The method of claim 76, wherein the cream formulation has a pH from about 2.8 to about 3.6.

78. The method of claim 1, further comprising administering to the patient an additional therapeutic agent.
